(19) United States
(12) Patent Application Publication
(10) Pub. No.: US 2014/0051129 A1
(43) Pub. Date: Feb. 20, 2014

(54) POTENTIATION OF ENZYMATIC SACCHARIFICATION

(71) Applicants: Jason Nichols, Research Triangle Park, NC (US); Brian Ember, Durham, NC (US); Paul Oeller, Research Triangle Park, NC (US); Myoung Kim, Research Triangle Park, NC (US)

(72) Inventors: Jason Nichols, Research Triangle Park, NC (US); Brian Ember, Durham, NC (US); Paul Oeller, Research Triangle Park, NC (US); Myoung Kim, Research Triangle Park, NC (US)

(73) Assignee: SYNGENTA PARTICIPATIONS AG, Research Triangle Park, NC (US)

(21) Appl. No.: 14/001,254

(22) PCT Filed: Feb. 22, 2012
(86) PCT No.: PCT/US12/25995

§ 371 Date: Nov. 4, 2013

Related U.S. Application Data
(60) Provisional application No. 61/445,616, filed on Feb. 23, 2011.

(51) Int. Cl. C12P 19/14

(52) U.S. Cl. CPC C12P 19/14
USPC 435/99

(57) ABSTRACT

The present disclosure provides methods of potentiating the activity of an enzyme cocktail by the addition of one or more enzymes. In some embodiments, a sub-maximum or sub-optimal dose of the cocktail may be used in combination with the enzymes. In some embodiments, the enzyme or enzymes are expressed in planta.

### RELATED APPLICATIONS

This application claims the benefit of U.S. Provisional Patent Application Ser. No. 61/445,616, filed Feb. 23, 2011, the disclosure of which is incorporated by reference herein in its entirety.

### FIELD OF THE INVENTION

The invention relates broadly to the field of enzymology and methods of making fermentable sugars.

### BACKGROUND OF THE INVENTION

In an effort to produce a more renewable fuel, there is considerable interest in the conversion of cellulosic biomass to fermentable sugars, which can then be used for a variety of purposes, including the production of ethanol. Cellulosic biomass includes commercially produced plant materials such as corn, soybean, vegetables and products from the wood-based industries, as well as waste products that contain cellulose such as cardboard and paper waste from a variety of sources. The conversion of cellulosic biomass to fermentable sugars, and the further conversion of these fermentable sugars to ethanol, is currently being pursued as a potential replacement for the use of fossil fuels as a source of energy.

To accomplish this chemical conversion of the cellulosic biomass, it is standard to use microbially-produced enzyme cocktails, which are heterogeneous mixtures of typically tens of different enzymes. However, due to the high cost required for production of these enzyme cocktails, coupled with the enormous amount of cellulolytic and/or hemicellulolytic enzymes required for a high percentage of conversion of cellulosic biomass to fermentable sugars, the conversion of cellulosic biomass to fermentable sugars is currently cost-prohibitive on a commercial scale. Therefore, strategies are needed to increase the efficiency and/or reduce the cost of this conversion process.

### SUMMARY OF THE INVENTION

The present disclosure provides methods of potentiating the activity of an enzyme cocktail by adding one or more additional enzymes. In some embodiments, a sub-maximum or sub-optimal dose of the enzyme cocktail may be used in combination with the enzymes provided herein, which combination surprisingly results in more effective enzymatic activity.

Provided are methods of potentiating enzymatic saccharification of a cellulosic biomass using a microbial enzyme cocktail, including combining the microbial enzyme cocktail with at least one cellulase enzyme (e.g., cellobiohydrolase I, cellobiohydrolase II and/or endoglucanase). In some embodiments, the microbial enzyme cocktail is provided at a sub-maximum dose (e.g., provided at a concentration of from 1, 5, 10, or 15, to 25, 30, 40, 50, 60, 70, 80 or 90 mg per gram of cellulose of the plant). In some embodiments, the cellulose enzyme or mixture of enzymes is provided at a concentration of from 0.05, 0.25, 1, 2.5, 5, 7, or 10, to 15, 20, 25, or 30 mg per gram of cellulose of the plant. In some embodiments, the enzyme or enzymes are expressed in piano. In some embodiments, the cellulase enzyme is provided by expression thereof by a transgenic plant (e.g., sorghum, maize, soybean, switchgrass, sugar cane, etc.) comprising a heterologous nucleic acid encoding the same.

In some embodiments, the biomass comprises said transgenic plant. In some embodiments, the transgenic plant is added to the biomass.

In some embodiments, the cellulosic biomass includes sugarcane bagasse, corn seed fiber, corn stover, switchgrass, wood pulp, or straw of rice, wheat or barley. In some embodiments, the cellulosic biomass is pretreated.

Further provided are methods of producing fermentable sugars, including: combining a cellulosic biomass with a composition comprising a microbial enzyme cocktail and at least one cellulase enzyme (e.g., cellobiohydrolase I, cellobiohydrolase II and/or endoglucanase) under conditions conducive to producing fermentable sugars therefrom, to thereby produce fermentable sugars. In some embodiments, the microbial enzyme cocktail is provided at a sub-maximum dose. In some embodiments, the methods further include a step of fermenting the fermentable sugars to produce ethanol.

Also provided are methods of producing fermentable sugars, including: (a) providing a mixture of: (i) a transgenic plant (e.g., sorghum, maize, soybean, switch grass, sugar cane, etc.) comprising a cellulase enzyme (e.g., cellobiohydrolase I, cellobiohydrolase II and/or endoglucanase) expressed from a heterologous nucleic acid encoding the same therein; and (ii) a composition comprising a microbial enzyme cocktail, and (b) providing conditions conducive to producing fermentable sugars from said mixture, to thereby produce the fermentable sugars.

In some embodiments, the transgenic plant expresses from 0.05, 0.25, 1, 2.5, 5, 7, or 10, to 15, 20, 25, or 30 mg of cellulase enzyme per gram of cellulose of said plant.

Further provided is the use of a cellulose enzyme or a transgenic plant transformed with a nucleic acid encoding the same as provided herein for the potentiation of enzymatic saccharification as described herein.

### BRIEF DESCRIPTION OF THE DRAWINGS

FIG. 1. Conversion of acid-steam exploded sugarcane bagasse to glucose by an enzyme cocktail (Accellerase® 1000) plus a set of three enzymes: CBH I+CBH II+EG (labeled as 3EC for three-enzyme mixture).

FIG. 2. Release of glucose from non-pretreated corn seed fiber catalyzed by a mixture of an enzyme cocktail and defined mixtures of hemicellulolytic enzymes.

FIG. 3. The ability of an enzyme cocktail (Accellerase® 1000) to convert ammonia treated cob/stover to fermentable sugars was enhanced by the addition of Xylanase, CBHI, and EG. No appreciable increase in cellulose conversion was observed in switchgrass samples, in the presence of plant-expressible enzymes. Cellulose conversion is normalized to the level achieved with 100 mg/g of the cocktail.

FIG. 4A-D. Cellulose conversion, normalized to level achieved with 100 mg/g cocktail (Accellerase® 1000), with respect to each component of the factorial screen: CBH I(A), EG(B), Xyl(C) or cocktail (D) with different combinations of enzymes for both cob/stover and switchgrass.

FIG. 5. The ability of enzyme cocktail (Accellerase® 1000) to convert steam-exploded bagasse to fermentable sugars is enhanced by the addition of both CBHI and EG. Cellulose conversion is normalized to level achieved with 100 mg/g cocktail.

FIG. 6A-E. Cellulose conversion, normalized to level achieved with 100 mg/g cocktail (Accellerase® 1000), with respect to each component of the factorial screen: CBH I(A), EG(B), LiP(C), bG(D) or cocktail (E) with different combinations of cellulase enzymes.

### DETAILED DESCRIPTION OF THE INVENTION

Various embodiments of the invention are described herein. As will be appreciated by those of skill in the art, the features of the various embodiments of the invention can be combined, creating additional embodiments which are intended to be within the scope of the invention. As used herein, “a,” “an” or “the” can mean one or more than one. For example, “a” cell is inclusive of a single enzyme as well as a multiplicity of enzymes. As used herein, “and/or” refers to and encompasses any and all possible combinations of one or more of the associated listed items, as well as the lack of combinations when interpreted in the alternative (“or”).

As used herein, “about” means within a statistically meaningful range of a value such as a stated concentration, time frame, weight (e.g., a percentage change (reduction or increase in weight)), volume, temperature or pH. Such a range can be within an order of magnitude, typically within 20%, more typically still within 10%, and even more typically within 5% of a given value or range. The allowable variation encompassed by “about” will depend upon the particular system under study, and can be readily appreciated by one of skill in the art.

The transitional phrase “consisting essentially of” means that the scope of a claim is to be interpreted to encompass the specified materials or steps recited in the claim “and those that do not materially affect the basic and novel characteristic(s)” of the claimed invention. Thus, the term “consisting essentially of” when used in a claim of this invention is not intended to be interpreted to be equivalent to “comprising.”

“Enzyme cocktail” as used herein refers to a heterogenous mixture of multiple enzymes produced and/or derived from an organism or organisms, such as bacteria and/or fungi, that are capable of breaking down cellulosic material. See, e.g., U.S. Patent Publication No. 2010/0086981 to LaTouf et al., which is incorporated by reference herein.

Enzyme cocktails for use in the enzymatic conversion of cellulosic biomass are commercially available from several companies. Examples include, Accellerase® 1000 and Accellerase® 1500, available from Genencor™ (Palo Alto, Calif.), and Cellic CTec2® from Novozymes™ (Franklinton, N.C.). These enzyme cocktails are largely produced from Trichoderma reesei and similar fungi, though not exclusively, and contain multiple enzymes with multiple functions, such as exoglucanases, endoglucanases, hemicellulases and beta-glucosidases.

| Enzyme Cocktail | Manufacturer | Species | Makeup | Optimal Dose |
|---|---|---|---|---|
| Accellerase ® | Genencor | Trichoderma | Multiple enzymes, | 0.1-0.5 mL/g |
| 1000 | | reesei | e.g., exoglucanases, | cellulose or |
| | | | endoglucanases, | 0.05-0.25 mL/g |
| | | | hemicellulases, beta | biomass |
| | | | glucosidases, etc. | |
| Accellerase ® | Genencor | Trichoderma | Multiple enzymes, | 0.1-0.5 mL/g |
| 1500 | | reesei | e.g., exoglucanases, | cellulose or |
| | | | endoglucanases, | 0.05-0.25 mL/g |
| | | | hemicellulases, beta | biomass |
| | | | glucosidases, etc. | |
| Accellerase ® | Genencor | | Multiple enzymes, | |
| DUET | | | including cellulases | |
| | | | and hemicellulases | |
| Celluclast ® | Novozymes | Trichoderma | | |
| 1.5 L | | reesei | | |
| Cellic CTec2 | Novozymes | | Cellulase complex | Suggested |
| | | | | dosage levels |
| | | | | for initial |
| | | | | investigation |
| | | | | are 1.5%-30.0% |
| | | | | w/w (g enzyme/g |
| | | | | cellulose) |
| Cellic HTec2 | Novozymes | | Endoxylanase | 0.05-0.50% |
| | | | cocktail | w/w (g enzyme/g |
| | | | | cellulose) |
| AlternaFuel ® | Dyadic | Trichoderma | fungal cellulase | Recommended |
| 200 P | | longibrachiatum | enzyme complex | to start with >0.01 g |
| | | | | per kg of original |
| | | | | biomass |
| AlternaFuel ® | Dyadic | Trichoderma | fungal hemicellulase | Recommended |
| 100 P | | longibrachiatum | enzyme complex | to start with >0.01 g |
| | | | | per kg of original |
| | | | | biomass |
| Ethazyme ™ | Zymetis | Saccharophagus | Cellulase and | |
| | | degradans | hemicellulase | |
| | | | complex cocktails | |

“Optimal dose” as used herein refers to a quantity of enzyme which is recommended by the manufacturer. For example, a particular dose or quantity of the Accellerase® enzyme cocktail is typically recommended by the manufacturer, Genencor™, for use in enzymatic saccharification. A “sub-optimal” dose is an amount or concentration of the enzyme which is less than that recommended, for example, 5, 10, 20, 10, 40 or 50% less than the recommended dosage.

“Maximum dose” refers to the lowest dosage at which there is maximum effect, or where a graph of the effect as a function of the concentration of cocktail begins to level off, in the absence of additional enzymes or other agents. For example, a maximum dose of the Accellerase® enzyme cocktail according to some embodiments is about 100 mg/g cellulose, the concentration at which a maximum effect is found, and thus is a maximum dose of that enzyme cocktail. A “sub-maximum” dose would be an amount or concentration of the enzyme which is less than the maximum dose, for example, 5, 10, 20, 30, 40, 50, 60, 70, 80 or 90% less than the maximum dose. For example, a sub-maximum dose includes 5, 10, 15, 20, 25, 30, 35, 40, 45, and 50 mg cocktail per g cellulose. It should also be noted that too high a dose of an enzyme cocktail, which often contains an ill-defined heterogeneous mixture of enzymes, may actually have an inhibitory effect.

Enzyme activity may be measured quantitatively using methods known in the art. For example, cellulase activity may be measured using Filter Paper Units per milliliter (FPU/ml) of original (undiluted) enzyme solution. Activity may also be measured using International Units (U) or Katal (KAT). A standard International Unit of enzyme activity (1 U) is defined as the amount of enzyme that catalyzes the formation of 1 μmol product, or conversion of 1 μmol of substrate, per minute. Katal is defined as the amount of enzyme that catalyzes the formation of 1 mol product per second. Thus, 1 Kat=6×10⁷ U.

“Potentiate”, “potentiating”, or grammatical variations thereof, refers to the increase or improvement in activity, whether additive or synergistic. For example, adding a substance such as a chemical or enzyme to a mixture which causes an apparent increase in the activity of the mixture (as compared to the activity without that chemical or enzyme) would be a method of potentiating the activity of the mixture. The mixture can contain, for example, a quantity of enzyme or enzyme cocktail, in addition to other components such as buffers, substrates such as biomass, or inert components.

“Cellulosic biomass” as used in the instant application refers to any cellulose containing substance. Examples include, but are not limited to, plant material such as non-edible plant material, including agricultural residues such as corn stover and sugarcane bagasse, plant material harvested for the purpose of converting the biomass to fermentable sugars, etc. Plant material can be from any plant, including, but not limited to, grasses or other monocots, dicots, or trees. In addition, cellulosic biomass includes waste products such as wood chips, shavings, pulp or other byproducts from the sawmill or paper making industries; or any post-consumer material, such as municipal paper waste (e.g., paper or cardboard), which contains cellulose.

The major components of terrestrial plants are represented by two families of sugar polymers: cellulose and hemicellulose. Cellulose fibers comprise 4%-50% of the total dry weight of plant stems, roots, and leaves. These fibers are embedded in a matrix of hemicellulose and phenolic polymers. Cellulose is a polymer composed of six-carbon sugars, mostly glucose, linked by β-1,4 linkages. Hemicellulose is a polymer of sugars, but the types of sugars vary with the source of biomass. With the exception of softwoods, the five-carbon sugar xylose is the predominant component in hemicellulose.

The compositions and methods described herein can aid in the processing of cellulosic biomass to many useful organic chemicals, fuels and products. For example, some commodity and specialty chemicals that can be produced from cellulosic biomass include, but are not limited to, acetone, acetate, butanediol, cis-muconic acid, ethanol, ethylene glycol, furfural, glycerol, glycine, lysine, organic acids (e.g., lactic acid), 1,3-propanediol, polyhydroxyalkanoates, and xylose. Likewise, animal feed and various food/beverages may be produced. See generally, Lynd et al. (1999) Biotechnol. Prog. 15:777-793; Philippidis, “Cellulose bioconversion technology” pp 179-212 In: Handbook on Bioethanol: Production and Utilization, ed. Wyman (Taylor & Francis 1996); and Ryu & Mandels (1980) Enz. Microb. Technol. 2:91-102.

“Saccharification” refers to the breaking of a complex carbohydrate, such as cellulose, into a smaller subunit, such as its monosaccharide components (e.g., a sugar such as glucose). For example, “enzymatic saccharification” may be used to convert cellulosic biomass to fermentable sugars through the use of at least one cellulose degrading enzyme therefor during any phase of the conversion process.

Enzymatic saccharification is typically a heterogeneous reaction that can be influenced by the structural features of the substrate. The process of converting cellulosic biomass to fermentable sugars can also involve several steps, which may include physically mixing the cellulosic biomass with chemicals such as solvents (e.g., acidic, basic or neutral solvents such as acids or bases or alcohols), exposing the cellulosic biomass to extreme conditions (e.g., high or low temperatures, and/or high or low atmospheric pressure), and/or combining the cellulosic biomass with enzymes which further break down the components of the cellulosic biomass to smaller elements. Enzymatic saccharification as used herein is intended to include any process for converting cellulosic biomass to fermentable sugars that incorporates at any point in the process an enzyme which is able to convert cellulose to a smaller subunit.

Enzymatic saccharification of cellulosic biomass can be accomplished with enzymes such as cellulases, which are able to bind and degrade cellulose. A wide variety of organisms, including bacteria and fungi, have cellulolytic activity by producing enzyme systems having multiple enzymes working synergistically to reduce cellulose to cellobiose, and then to glucose and/or other sugars.

“Cellulase enzyme” as used herein refers to an enzyme capable of degrading cellulose. There are three main types of cellulase enzymes, which are not to be limiting of the invention: cellobiohydrolases (CBH) (also known as exoglucanases), endoglucanases (EG), and beta-glucosidases. In some applications, use of all three types of cellulases results in synergistic hydrolysis. However, the relative amount of each enzyme in a given cellulase preparation is dependent upon the source of the enzymes, and the mechanisms of this synergistic action are poorly understood. In some embodiments, enzymes are thermostable (i.e., active at high temperatures).

Cellobiohydrolases (CBH) cleave cellobiose from either the reducing or the non-reducing end of a cellulose chain, and include Cellobiohydrolase I (CBH I) enzymes (also known as Cel7a) and Cellobiohydrolase II (CBH II) enzymes (also known as Cel6). CBH enzymes have a distinct structure, which includes an amino terminal catalytic domain which is greater than 50% of the molecule, a flexible and glycosylated linker domain and a carboxy terminal domain which is the cellulose binding domain. CBH enzymes are processive enzymes, meaning that they typically attach to an end of the cellulose fiber and processively move along the cellulose fiber. These enzymes are active on crystalline cellulose, as well as on amorphous (loose, open or random structure) cellulose. CBH enzymes have been isolated from a variety of sources, including microbial sources such as bacteria, yeast, and fungi, each of which is encompassed herein, as well as homologues thereof.

Cellobiohydrolase I (CBH I) enzymes belong to the glycosyl hydrolase family 7 enzymes. These enzymes release cellobiose (β1,4 linked glucose disaccharides) from the reducing end of the cellulose chain. The CBH I enzymes are only derived from fungi; however, some bacteria produce a CBH I-like molecule which has a similar function (meaning that it acts from the reducing end of a cellulose fiber to release cellobiose). The bacterial enzymes with CBH I-like activity belong to glycosyl hydrolase family 48 enzymes and are also known as Cel48 enzymes.

Cellobiohydrolase II (CBH II) enzymes, also known as Cel6 enzymes or exo-cellulases, belong to the glycosyl hydrolase family 6 enzymes. These enzymes release cellobiose from the non-reducing end of the cellulose chain. CBH II enzymes have been isolated from fungi as well as other microbial sources.

Endoglucanases (EG), which include EG I through EG IV, typically work by randomly cleaving β1-4 glycosidic internal bonds on the cellulose chains. EG works primarily on amorphous (loose, open or random structure) cellulose. EG are produced by a broad range of organisms, including fungi, bacteria, plants, and insects, each of which is encompassed herein, as well as homologues thereof.

Beta-glucosidases are exocellulases that cleave terminal β1-4 glucose bonds to release glucose.

Other enzymes which may be used to aid in cellulosic conversion include hemicellulases (e.g., xylanase), which break bonds in the backbone chain of hemicellulose, and lignin modigyin enzymes (LME) (e.g., laccase, lignin peroxidase (LiP), and magnanese peroxidase (MnP)), which are involved in lignin degradation.

Enzymes may be isolated from natural sources or may be produced using recombinant hosts (e.g., bacterial, fungal, plant) as known in the art. For example, heterologous expression of endoglucanases, exoglucanases, and β-D-glucosidases in E. coli, Bacillus subtilis, and Streptomyces lividans have been reported (Lejeune et al., Biosynthesis and Biodegradation of Cellulose; Haigler, C. H.; Weimer, P. J., Eds.; Marcel Dekker: New York, N.Y., 1990; pp. 623-671). In addition, the expression of a B. subtilis endoglucanase and a C. fimi β-D-glucosidase in E. coli has been demonstrated (Yoo et al. (1992) Biotechnol. Lett. 14:77-82). Expression of the enzymes in plants is also known, as well as described hereinbelow.

CBH I may include, for example, the amino acid sequence of SEQ ID NO:1 (CBH I plus N-terminal signal sequence), a functional fragment thereof (e.g., CBH I without the signal sequence or a functional fragment thereof), or a protein having at least 80, 90, 95 or 99% identity thereto. CBH II may be, for example, the amino acid sequence of SEQ ID NO:2 (CBH II plus N-terminal signal sequence), a functional fragment thereof (e.g., CBH II without the signal sequence or a functional fragment thereof), or a protein having at least 80, 90, 95 or 99% identity thereto. EG may be, for example, the amino acid sequence of SEQ ID NO:3, a functional fragment thereof, or a protein having at least 80, 90, 95 or 99% identity thereto, or the amino acid sequence of SEQ ID NO:4, a functional fragment thereof, or a protein having at least 80, 90, 95 or 99% identity thereto. Functional fragments may be at least 100, 150, 200, 250, 300, 350, 400, 450, 500, 550, 600, 650, or 700 consecutive amino acids according to some embodiments.

The cellulase enzymes may be used alone or in combination in the methods taught herein, with the ratios of various enzymes and/or enzyme cocktails may be optimized by one of skill in the art as desired for maximum saccharification without causing the competitive inhibition over substrate sites.

In some embodiments, at least about 1, 5 or 10, and/or up to about 15, 20, 25, or 30 mg cellulase enzyme per g cellulose may be used, alone or in combination with that amount of other cellulase enzymes, to potentiate the activity of a microbial enzyme cocktail. In some embodiments, the microbial enzyme cocktail is provided at concentrations of from about 5, 10, 15, 20, 25, or 30, to about 40, 50, 60, or 70 mg enzyme cocktail per g cellulose.

If desired, biomass may also be “pretreated,” as known in the art, by chemical (e.g., acid or alkali) treatment, mechanical treatment (e.g., grinding), etc., prior to or during saccharification. The pretreatment may be a chemical treatment involving the addition of an acid or alkali which alters the pH of the biomass to disrupt its fiber structure and increase its accessibility or susceptibility to being hydrolyzed in a subsequent enzymatic hydrolysis.

Mechanical pretreatment typically includes the use of pressure, grinding, milling, agitation, shredding, compression/expansion and chemical action may include the use of heat (often steam), acid or alkali, or solvents.

Pretreatment with acid can aid in hydrolyzing the hemicellulose, or a portion thereof, that is present in the biomass to the monomeric sugars xylose, arabinose, mannose, galactose, or a combination thereof. Typically, a dilute acid (e.g., at a concentration from about 0.02% (w/w) to about 2% (w/w), or any amount therebetween, measured as the percentage weight of pure acid in the total weight of dry feedstock plus aqueous solution) is employed for the pretreatment. In some embodiments, the acid pretreatment is carried out at a peak temperature of about 160° C. to about 280° C. for a time of about 6 seconds to about 600 seconds, at a pH of about 0.4 to about 2.0. It should be understood that the acid pretreatment may be carried out in more than one stage, although in some embodiments it is performed in a single stage.

One method of performing acid pretreatment of the feedstock is steam explosion, for example, using the process conditions described in U.S. Pat. No. 4,461,648 (Foody, which is herein incorporated by reference). The pretreatment may be a continuous process, for example, as described in U.S. Pat. No. 5,536,325 to Brink, WO 2006/128304 to Foody et al., U.S. Pat. No. 4,237,226 to Grethlein, etc., which are each incorporated herein by reference. Other techniques that are known in the art and that may be used include, but are not limited to, those disclosed in U.S. Pat. No. 4,556,430 to Converse et al., which is incorporated herein by reference.

Ammonia or ammonium hydroxide may be used for alkali pretreatment of the biomass. Pretreatment with ammonia or ammonium hydroxide reacts with acidic groups present on the hemicellulose to open up the surface of the substrate and may or may not hydrolyze the hemicellulose component of the feedstock. The addition of the alkali may also alter the crystal structure of the cellulose so that it is more amenable or susceptible to hydrolysis.

An example of a suitable alkali pretreatment, variously called Ammonia Freeze Explosion, Ammonia Fiber Explosion or Ammonia Fiber Expansion (“AFEX” process), involves contacting the lignocellulosic feedstock with ammonia or ammonium hydroxide in a pressure vessel for a sufficient time to enable the ammonia or ammonium hydroxide to alter the crystal structure of the cellulose fibers. The pressure is then rapidly reduced, which allows the ammonia to flash or boil and explode the cellulose fiber structure. The flashed ammonia may then be recovered according to known processes. Another suitable alkali pretreatment for use in the present invention employs dilute solutions of ammonium hydroxide. Treatment of biomass with alkali is disclosed in U.S. Pat. Nos. 5,171,592, 5,037,663, 4,600,590, 6,106,888, 4,356,196, 5,939,544, 6,176,176, 5,037,663 and 5,171,592, US2009/0053770 and US2007/0031918, which are each incorporated herein by reference.

In some embodiments, saccharification of a cellulosic biomass yields sugars that can be fermented to produce a fermentation broth containing alcohol. “Fermentable sugar” is a sugar capable of fermentation, the process of deriving energy from the oxidation of organic compounds, to produce useful substances such as ethanol. For example, glucose products may be fermented to provide ethanol using a yeast (e.g., Saccharomyces cerevisiae). Examples of fermentable sugars include, but are not limited to, glucose, xylose, pentose, and hexose.

For ethanol production, the fermentation is typically carried out with a Saccharomyces spp. yeast. In some embodiments, glucose and any other hexoses typically present in the hydrolysate slurry are fermented to ethanol by wild-type Saccharomyces cerevisiae, although genetically modified yeasts may be employed, as well. For example, the fermentation may be performed with a recombinant Saccharomyces yeast that is engineered to ferment both hexose and pentose sugars to ethanol. Recombinant yeasts that can ferment the pentose sugar, xylose, to ethanol are described in U.S. Pat. No. 5,789,210, which is incorporated by reference herein. Furthermore, the pentose sugars, arabinose and xylose, may be converted to ethanol by the yeasts described in Boles et al. (WO 2006/096130, which is incorporated herein by reference).

Examples of other alcohol fermentation products include, but are not limited to, butanol, 1,3-propanediol and 2,3-butanediol. Alcohols may be extracted from the fermentation broth by a solvent and then concentrated by distilling the mixture of alcohol and solvent to produce an alcohol-enriched vapour. Additional examples of microorganisms that may be employed in the fermentation include wild-type or recombinant Escherichia, Zymomonas, Candida, Pichia, Streptomyces, Bacillus, Lactobacillus and Clostridium.

### Enzyme Expression in Plants

In some embodiments, expression of one or more of the enzymes is done in planta, making the enzyme(s) conveniently available for potentiation of the enzymatic saccharification of the plant biomass, alone or in combination with other biomass, upon addition of an enzyme cocktail. Methods for expressing cellulase enzymes in plants is known and described in, for example, U.S. Pat. No. 7,361,806 to Lebel et al., U.S. Patent Application Publication Nos. 2009/0203079 and 2008/0118954 to Sticklen, 2007/0250961 to Blaylock et al., 2009/0205086 to Hood et al., and 2009/0193541 to Miles, the disclosures of which are incorporated by reference herein in their entireties. This may be accomplished by expression of a heterologous nucleic acid encoding such enzymes. See U.S. Patent Application Publication No. 2010/0189706 to Chang et al., which is incorporated by reference herein in its entirety.

The terms “heterologous” and “exogenous” when used herein to refer to a nucleic acid sequence (e.g. a DNA or RNA sequence) or a gene, refer to a sequence that originates from a source foreign to the particular host cell or, if from the same source, is modified from its original form. The terms “heterologous” and “exogenous” also include non-naturally occurring multiple copies of a naturally occurring DNA sequence. Thus, the terms refer to a nucleic acid (e.g., DNA) segment that is foreign to the cell. “Expression” of a nucleic acid as used herein refers to the transcription, and preferably, translation thereof.

It should be understood that the enzymes may be expressed in the plants intended to be used as feedstocks for fermentable sugar production, and/or may be expressed by plants that are harvested and added (the transgenic plant or a part thereof containing the expressed enzyme) to a biomass to be converted to fermentable sugars. In other embodiments, the plant expressed enzyme may be extracted from the transgenic plant or plant part thereof and added to a composition comprising the biomass and/or enzyme cocktail.

The expressed enzyme may be targeted to a particular plant tissue or tissues, or to a particular intracellular compartment or compartments, e.g., the vacuoles, using routine methods. Plant vacuoles represent the largest compartment in the plant cell for dissolved substances. The most important storage proteins of tubers, bulbs, roots and stems, for example, are located in the vacuoles of the cells that compose those organs. Moreover, the storage proteins of most seeds are located in so-called protein bodies, which are specialized vacuoles to which the same sorting signals would seem to apply as to the vacuoles of the vegetative organs.

Targeting expressed multidomain enzymes to certain organelles such as vacuoles according to some embodiments may alleviate toxicity problems. For vacuole-targeted expression of multidomain enzymes, plants can be transformed with vectors that include a vacuolar targeting sequence, such as that from a tobacco chitinase gene. In this case, the expressed multidomain enzyme is stored in the vacuoles where they will not be able to degrade cellulose and harm the plant. In some embodiments, the vacuole sorting signal sequence is derived from the barley polyamino oxidase 2 (BPAO2) signal sequence. BPAO2 has an N-terminal signal peptide for entry into the secretory pathway. The presence of a C-terminal extension of this signal peptide results in vacuolar localization of BPAO in a plant cell (see Cervelli et at (2004) The Plant Journal 40:410-418). In another embodiment, useful vacuole sorting signals are described in U.S. Application Publication No. 2009/0193541, which is herein incorporated by reference in its entirety.

In various embodiments of the present invention, modified multidomain enzyme coding sequences may be fused to promoters active in plants and transformed into the nuclear genome or the plastid genome, Chloroplast expression has the advantage that the multidomain enzyme is less damaging to the plastid as it contains little or no cellulose.

A “crop plant” is any plant that is cultivated for the purpose of producing plant material that is sought after by man for either oral consumption, or for utilization in an industrial, pharmaceutical, or commercial process. Any of a variety of plants, including monocots and dicots, may be used to express one or more enzymes such as cellulases, including, but not limited to maize, wheat, rice, barley, soybean, cotton, sorghum, oats, tobacco, Miscanthus grass, Switch grass, trees, beans in general, rape/canola, alfalfa, flax, sunflower, safflower, millet, rye, sugarcane (including energy cane), sugar beet, cocoa, tea, Brassica, cotton, coffee, sweet potato, flax, peanut, clover; vegetables such as lettuce, tomato, cucurbits, cassava, potato, carrot, radish, pea, lentils, cabbage, cauliflower, broccoli, Brussels sprouts, peppers, and pineapple; tree fruits such as citrus, apples, pears, peaches, apricots, walnuts, avocado, banana, and coconut; and flowers such as orchids, carnations and roses.

In some embodiments, the enzymes may be modified to include a heterologous linker region that improves the expression, stability and/or activity of the multi-domain plant expressed enzyme, for example, a cellulase enzyme such as CBH I, CBH II or EG. See, e.g., PCT Patent Application Publication No. WO 2010/091149, the disclosure of which is incorporated by reference herein in its entirety. Cellobiohydrolases and endoglucanases are structurally similar and are frequently composed of multiple domains. At least one of the domains is a catalytic core domain which may be associated with additional catalytic domains or at least one cellulose-binding domain (CBD). In some embodiments, the two domains can be connected by relatively long, glycosylated linker peptides of 6-59 amino acids. The heterologous linker sequence may be resistant to cleavage by a plant protease due to the replacement of protease sensitive sites with protease insensitive sites or by altering the structural conformation of the multidomain enzyme such that protease-sensitive sites are inaccessible to the plant proteases.

The modified cellulase enzymes according to these embodiments may have a linker sequence that results in less cleavage when the modified cellulase is expressed in plants as compared with the amount of cleavage detected with a native linker sequence. In some embodiments, less than about 90% of the modified enzyme is cleaved when expressed in plants, less than about 80%, less than about 70%, less than about 60%, less than about 50%, less than about 40%, less than about 30%, less than about 20%, less than about 10%, less than about 5%, or none of the modified enzyme is cleaved when expressed in plants. The heterologous linker sequence may result in less cleavage due to the replacement or protection of protease-sensitive cleavage sites as discussed supra. Thus, the modified cellulase may have improved expression, stability, and/or activity relative to a control cellulase.

The modified multidomain enzyme according to some embodiments may be composed of at least one first domain, at least one heterologous linker, and at least one second domain. In some embodiments, the first domain and/or the second domain may be non-heterologous sequences. By “non-heterologous” it is intended that the first domain and the second domain are derived from the same native multidomain enzyme and may contain minor modifications which result in a domain polypeptide sequence which is greater than about 80% identical, greater than about 85% identical, greater than about 90% identical, greater than about 95% identical, greater than about 96% identical, greater than about 97% identical, greater than about 98% identical, or greater than about 99% identical to the native polypeptide sequence. The structure of many cellulases is described in Gilkes et al. (1991) Microbiological Reviews 55(2):303-315, which is herein incorporated by reference in its entirety.

As taught herein, the addition of transgenic plants or plant parts containing plant expressed cellulase enzymes may increase the efficiency of conventional cellulose degradation processes that make use of enzyme cocktails. This may be through the addition of transgenic plants or plant parts to a mixture of biomass (e.g., a plant material that is not transgenic), which results in a increase in the overall efficiency and/or yield in the production of fermentable sugars from the biomass as compared to that without the plant expressed cellulase enzyme(s), particularly in the presence of an enzyme cocktail used for the biomass conversion. In some embodiments, the enzymatic digestion of the biomass may be carried out using a lower amount of the cocktails.

Also provided are compositions comprising a biomass, which may comprise a transgenic plant or plant part comprising a plant expressed cellulose, and an enzyme cocktail, and in some embodiments the compositions are provided with conditions conducive to enzymatic conversion of the biomass to fermentable sugars using the cellulase and/or enzyme cocktail. Nucleic acid sequences useful for transformation and expression of a multidomain enzyme in a plant cell of interest are known and described in, for example, the patent application publications noted above, which are incorporated by reference herein.

The nucleic acid sequences may be present in nucleic acid constructs (e.g., DNA or RNA) such as expression cassettes. “Expression cassette” as used herein means a nucleic acid molecule capable of directing expression of a particular nucleotide sequence in an appropriate host cell, generally comprising a promoter operatively linked to the nucleotide sequence of interest (i.e., a nucleotide sequence encoding a polypeptide of interest) which is operatively linked to termination signals. It also typically comprises sequences required for proper translation of the nucleotide sequence. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. Typically, however, the expression cassette is heterologous with respect to the host, i.e., the particular DNA sequence of the expression cassette does not occur naturally in the host cell and must have been introduced into the host cell or an ancestor of the host cell by a transformation event. The expression of the nucleotide sequence in the expression cassette may be under the control of a constitutive promoter or of an inducible promoter that initiates transcription primarily when the host cell is exposed to some particular external stimulus. Additionally, the promoter can also be specific or show a preferential expression for a particular tissue or organ or stage of development.

In addition, the construct may further comprise additional regulatory elements to facilitate transcription, translation, or transport of an expressed enzyme. The regulatory sequences of the expression construct are operably linked to the polynucleotide encoding the enzyme. By “operably linked” is intended a functional linkage between a regulatory element and a second sequence wherein the regulatory element initiates and/or mediates transcription, translation, or translocation of the nucleic acid sequence corresponding to the second sequence. Generally, operably linked means that the nucleotide sequences being linked are contiguous; however, the presence of intervening sequence between the regulatory elements is not intended to mean that the elements are not contiguous. The regulatory elements include promoters, enhancers, and signal sequences useful for targeting cytoplasmically-synthesized proteins to the endomembrane system of the plant cell.

In some embodiments, the construct comprises, in the 5′ to 3′ direction of transcription, a transcriptional and translational initiation region (i.e., a promoter), a polynucleotide encoding an endoplastic reticulum signal sequence, and a polynucleotide encoding the enzyme. Exemplary signal sequences include the SEKDEL (SEQ ID NO:5) endoplasmic reticulum targeting sequence, the gamma zein 27 kD signal sequence, and the Glycine max glycinin GY1 signal sequence. Other signal sequences useful in the methods of the invention will be apparent to one of skill in the art.

Any promoter capable of driving expression in the plant of interest may be used in the practice of the invention. The promoter may be native or analogous or foreign or heterologous to the plant host.

The choice of promoters to be included depends upon several factors, including, but not limited to, efficiency, selectability, inducibility, desired expression level, and cell- or tissue-preferential expression. It is a routine matter for one of skill in the art to modulate the expression of a sequence by appropriately selecting and positioning promoters and other regulatory regions relative to that sequence.

Some suitable promoters initiate transcription only, or predominantly, in certain cell types. Thus, as used herein a cell type- or tissue-preferential promoter is one that drives expression preferentially in the target tissue, but may also lead to some expression in other cell types or tissues as well. Methods for identifying and characterizing promoter regions in plant genomic DNA include, for example, those described in the following references: Jordano, et al., Plant Cell, 1:855-866 (1989); Bustos, et al., Plant Cell, 1:839-854 (1989); Green, et al., EMBO J. 7, 4035-4044 (1988); Meier, et al., Plant Cell, 3, 309-316 (1991); and Zhang, et al., Plant Physiology 110: 1069-1079 (1996).

Promoters active in photosynthetic tissue that preferentially drive transcription in green tissues such as leaves and stems are also of interest for use in plant expression. For example, the promoter may drive expression only or predominantly in such tissues. The promoter may confer expression constitutively throughout the plant, or differentially with respect to the green tissues, or differentially with respect to the developmental stage of the green tissue in which expression occurs, or in response to external stimuli.

Examples of such promoters include the ribulose-1,5-bisphosphate carboxylase (RbcS) promoters such as the RbcS promoter from eastern larch (Larix laricina), the pine cab6 promoter (Yamamoto et al. (1994) Plant Cell Physiol. 35:773-778), the Cab-1 gene promoter from wheat (Fejes et al. (1990) Plant Ma Biol. 15:921-932), the CAB-1 promoter from spinach (Lubberstedt et al. (1994) Plant Physiol. 104:997-1006), the cab1R promoter from rice (Luan et al. (1992) Plant Cell 4:971-981), the pyruvate orthophosphate dikinase (PPDK) promoter from corn (Matsuoka et al. (1993) Proc Natl Acad Sci USA 90:9586-9590), the tobacco Lhcb1*2 promoter (Cerdan et al. (1997) Plant Mol. Biol. 33:245-255), the Arabidopsis thaliana SUC2 sucrose-H+ symporter promoter (Truernit et al. (1995) Planta 196:564-570), and thylakoid membrane protein promoters from spinach (psaD, psaF, psaE, PC, FNR, atpC, atpD, cab, rbcS). Other promoters that drive transcription in stems, leafs and green tissue are described in U.S. Patent Publication No. 2007/0006346, herein incorporated by reference in its entirety.

A maize gene encoding phosphoenol carboxylase (PEPC) has been described by Hudspeth & Grula (Plant Molec Biol 12: 579-589 (1989)). Using standard molecular biological techniques the promoter for this gene can be used to drive the expression of any gene in a green tissue-preferred manner in transgenic plants.

In some other embodiments of the present invention, inducible promoters may be desired. Inducible promoters primarily drive transcription in response to external stimuli such as chemical agents or environmental stimuli. For example, inducible promoters can confer transcription in response to hormones such as giberellic acid or ethylene, or in response to light or drought. With a chemically inducible promoter, expression of the enzyme genes transformed into plants may be activated at an appropriate time by foliar application of a chemical inducer.

A variety of transcriptional terminators are available for use in expression cassettes. These are responsible for the termination of transcription beyond the transgene and correct mRNA polyadenylation. The termination region may be native with the transcriptional initiation region, may be native with the operably linked DNA sequence of interest, may be native with the plant host, or may be derived from another source (i.e., foreign or heterologous to the promoter, the DNA sequence of interest, the plant host, or any combination thereof). Appropriate transcriptional terminators are those that are known to function in plants and include the CAMV 35S terminator, the tml terminator, the nopaline synthase terminator and the pea rbcs E9 terminator. These can be used in both monocotyledons and dicotyledons. In addition, a gene's native transcription terminator may be used.

In some embodiments, the expression cassette will comprise a selectable marker gene for the selection of transformed cells. Selectable marker genes are utilized for the selection of transformed cells or tissues.

Various intron sequences have been shown to enhance expression, particularly in monocotyledonous cells. For example, the introns of the maize Adhl gene have been found to significantly enhance the expression of the wild-type gene under its cognate promoter when introduced into maize cells. Intron 1 was found to be particularly effective and enhanced expression in fusion constructs with the chloramphenicol acetyltransferase gene (Callis et al., Genes Develop. 1: 1183-1200 (1987)). In the same experimental system, the intron from the maize bronze 1 gene had a similar effect in enhancing expression. Intron sequences have been routinely incorporated into plant transformation vectors, typically within the non-translated leader.

A number of non-translated leader sequences derived from viruses are also known to enhance expression, for example, in dicotyledonous cells. Specifically, leader sequences from Tobacco Mosaic Virus (TMV, the “W-sequence”), Maize Chlorotic Mottle Virus (MCMV), and Alfalfa Mosaic Virus (AMV) have been shown to be effective in enhancing expression (e.g. Gallie et al. Nucl. Acids Res. 15: 8693-8711 (1987); Skuzeski et al. Plant Molec. Biol. 15: 65-79 (1990)). Other leader sequences known in the art include but are not limited to: picornavirus leaders, for example, EMCV leader (Encephalomyocarditis 5′ noncoding region) (Elroy-Stein, O., Fuerst, T. R., and Moss, B. PNAS USA 86:6126-6130 (1989)); potyvirus leaders, for example, TEV leader (Tobacco Etch Virus) (Allison et al., 1986); MDMV leader (Maize Dwarf Mosaic Virus); Virology 154:9-20); human immunoglobulin heavy-chain binding protein (BiP) leader, (Macejak, D. G., and Samow, P., Nature 353: 90-94 (1991); untranslated leader from the coat protein mRNA of alfalfa mosaic virus (AMV RNA 4), (Jobling, S. A., and Gehrke, L., Nature 325:622-625 (1987); tobacco mosaic virus leader (TMV), (Gallie, D. R. et al., Molecular Biology of RNA, pages 237-256 (1989); and Maize Chlorotic Mottle Virus leader (MCMV) (Lommel, S. A. et al., Virology 81:382-385 (1991). See also, Della-Cioppa et al., Plant Physiology 84:965-968 (1987).

It will also be recognized that the nucleotide sequence encoding the enzyme may be optimized for increased expression in the transformed host cell. For example, the nucleotide sequences can be synthesized using host cell-preferred codons for improved expression, or may be synthesized using codons at a host-preferred codon usage frequency. Generally, the GC content of the gene will be increased. See, for example, Campbell and Gown (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

The expression constructs described herein can be introduced into the plant cell in a number of art-recognized ways. The term “introducing” in the context of a polynucleotide, for example, a nucleotide construct of interest, is intended to mean presenting to the plant the polynucleotide in such a manner that the polynucleotide gains access to the interior of a cell of the plant. Where more than one polynucleotide is to be introduced, these polynucleotides can be assembled as part of a single nucleotide construct, or as separate nucleotide constructs, and can be located on the same or different transformation vectors. Accordingly, these polynucleotides can be introduced into the host cell of interest in a single transformation event, in separate transformation events, or, for example, in plants, as part of a breeding protocol. The methods of the invention do not depend on a particular method for introducing one or more polynucleotides into a plant, only that the polynucleotide(s) gains access to the interior of at least one cell of the plant. Methods for introducing polynucleotides into plants are known in the art including, but not limited to, transient transformation methods, stable transformation methods, and virus-mediated methods.

“Transient transformation” in the context of a polynucleotide is intended to mean that a polynucleotide is introduced into the plant and does not integrate into the genome of the plant.

By “stably introducing” or “stably introduced” in the context of a polynucleotide introduced into a plant is intended the introduced polynucleotide is stably incorporated into the plant genome, and thus the plant is stably transformed with the polynucleotide. In representative methods, “stable transformation” or “stably transformed” is intended to mean that a polynucleotide, for example, a nucleotide construct described herein, introduced into a plant integrates into the genome of the plant and is capable of being inherited by the progeny thereof, more particularly, by the progeny of multiple successive generations.

Numerous transformation vectors available for plant transformation are known to those of ordinary skill in the plant transformation arts, and the nucleic acids encoding the cellulase enzymes pertinent to this invention can be used in conjunction with any such vectors. The selection of vector will depend upon the preferred transformation technique and the target species for transformation. For certain target species, different antibiotic or herbicide selection markers may be preferred. Selection markers used routinely in transformation include the nptll gene, which confers resistance to kanamycin and related antibiotics (Messing & Vierra. Gene 19: 259-268 (1982); Bevan et al., Nature 304:184-187 (1983)), the bar gene, which confers resistance to the herbicide phosphinothricin (White et al., Nucl. Acids Res 18: 1062 (1990), Spencer et al. Theor. Appl. Genet 79: 625-631 (1990)), the hph gene, which confers resistance to the antibiotic hygromycin (Blochinger & Diggelmann, Mol Cell Biol 4: 2929-2931), and the dhfr gene, which confers resistance to methatrexate (Bourouis et al., EMBO J. 2(7): 1099-1104 (1983)), the EPSPS gene, which confers resistance to glyphosate (U.S. Pat. Nos. 4,940,935 and 5,188,642), and the mannose-6-phosphate isomerase gene, which provides the ability to metabolize mannose (U.S. Pat. Nos. 5,767,378 and 5,994,629).

Methods for transformation of plants are also well known in the art. For example, Ti plasmid vectors have been utilized for the delivery of foreign DNA, as well as direct DNA uptake, liposomes, electroporation, microinjection, and microprojectiles. In addition, bacteria from the genus Agrobacterium can be utilized to transform plant cells. Below are descriptions of representative techniques for transforming both dicotyledonous and monocotyledonous plants, as well as a representative plastid transformation technique.

Many vectors are available for transformation using Agrobacterium tumefaciens. These typically carry at least one T-DNA border sequence and include vectors such as pBIN19 (Bevan, Nucl. Acids Res. (1984)). For the construction of vectors useful in Agrobacterium transformation, see, for example, US Patent Application Publication No. 2006/0260011, herein incorporated by reference.

Transformation without the use of Agrobacterium tumefaciens circumvents the requirement for T-DNA sequences in the chosen transformation vector and consequently vectors lacking these sequences can be utilized in addition to vectors such as the ones described above which contain T-DNA sequences. Transformation techniques that do not rely on Agrobacterium include transformation via particle bombardment, protoplast uptake (e.g. PEG and electroporation) and microinjection. The choice of vector depends largely on the preferred selection for the species being transformed. For the construction of such vectors, see, for example, US Application No. 20060260011, herein incorporated by reference.

For expression of a nucleotide sequence of the present invention in plant plastids, an exemplary plastid transformation vector pPH143 (WO 97/32011, example 36) may be used. The nucleotide sequence may be inserted into pPH143 thereby replacing the PROTOX coding sequence. This vector is then used for plastid transformation and selection of transformants for spectinomycin resistance. Alternatively, the nucleotide sequence may be inserted in pPH143 so that it replaces the aadH gene. In this case, transformants are selected for resistance to PROTOX inhibitors.

Transformation techniques for dicotyledons are well known in the art and include Agrobacterium-based techniques and techniques that do not require Agrobacterium. Non-Agrobacterium techniques involve the uptake of exogenous genetic material directly by protoplasts or cells. This can be accomplished by PEG or electroporation mediated uptake, particle bombardment-mediated delivery, or microinjection. Examples of these techniques are described by Paszkowski et al., EMBO J. 3: 2717-2722 (1984), Potrykus et al., Mol. Gen. Genet, 199: 169-177 (1985), Reich et al., Biotechnology 4: 1001-1004 (1986), and Klein et al., Nature 327: 70-73 (1987). In each case, the transformed cells may be regenerated to whole plants using standard techniques known in the art.

Agrobacterium-mediated transformation is often used for transformation of dicotyledons because of its high efficiency of transformation and its broad utility with many different species. Agrobacterium transformation typically involves the transfer of the binary vector carrying the foreign DNA of interest (e.g. pCIB200 or pCIB2001) to an appropriate Agrobacterium strain which may depend of the complement of vir genes carried by the host Agrobacterium strain either on a co-resident Ti plasmid or chromosomally (e.g. strain CIB542 for pCIB200 and pCIB2001 (Uknes et al. Plant Cell 5: 159-169 (1993)). The transfer of the recombinant binary vector to Agrobacterium can be accomplished by a triparental mating procedure using E. coli carrying the recombinant binary vector, a helper E. coli strain which carries a plasmid such as pRK2013 and which is able to mobilize the recombinant binary vector to the target Agrobacterium strain. Alternatively, the recombinant binary vector can be transferred to Agrobacterium by DNA transformation (Hofgen & Willmitzer, Nucl. Acids Res. 16: 9877 (1988)).

Transformation of the target plant species by recombinant Agrobacterium usually involves co-cultivation of the Agrobacterium with explants from the plant and follows protocols well known in the art. Transformed tissue is regenerated on selectable medium carrying the antibiotic or herbicide resistance marker present between the binary plasmid T-DNA borders.

Another approach to transforming plant cells with a nucleic acid involves propelling inert or biologically active particles at plant tissues and cells. This technique is disclosed in, for example, U.S. Pat. Nos. 4,945,050, 5,036,006, and 5,100,792 all to Sanford et al. Generally, this procedure involves propelling inert or biologically active particles at the cells under conditions effective to penetrate the outer surface of the cell and afford incorporation within the interior thereof. When inert particles are utilized, the vector can be introduced into the cell by coating the particles with the vector containing the desired nucleic acid. Alternatively, the target cell can be surrounded by the vector so that the vector is carried into the cell by the wake of the particle. Biologically active particles (e.g., dried yeast cells, dried bacterium or a bacteriophage, each containing DNA sought to be introduced) can also be propelled into plant cell tissue.

Transformation of most monocotyledon species has now also become routine. Suitable techniques include direct gene transfer into protoplasts using PEG or electroporation techniques, and particle bombardment into callus tissue. Transformations can be undertaken with a single DNA species or multiple DNA species (i.e. co-transformation) and both of these techniques are suitable for use with this invention. Co-transformation may have the advantage of avoiding complete vector construction and of generating transgenic plants with unlinked loci for the gene of interest and the selectable marker, enabling the removal of the selectable marker in subsequent generations, should this be regarded desirable. However, a disadvantage of the use of co-transformation is the less than 100% frequency with which separate DNA species are integrated into the genome (Schocher et al. Biotechnology 4: 1093-1096 (1986)).

Patent Applications EP 0 292 435, EP 0 392 225, and WO 93/07278 describe techniques for the preparation of callus and protoplasts from an elite inbred line of maize, transformation of protoplasts using PEG or electroporation, and the regeneration of maize plants from transformed protoplasts. Gordon-Kamm et al. (Plant Cell 2: 603-618 (1990)) and Fromm et al. (Biotechnology 8: 833-839 (1990)) have published techniques for transformation of A188-derived maize line using particle bombardment. Furthermore, WO 93/07278 and Koziel et al. (Biotechnology 11: 194-200 (1993)) describe techniques for the transformation of elite inbred lines of maize by particle bombardment. This technique utilizes immature maize embryos of 1.5-2.5 mm length excised from a maize ear 14-15 days after pollination and a PDS-1000He Biolistics device for bombardment.

Transformation of rice can also be undertaken by direct gene transfer techniques utilizing protoplasts or particle bombardment. Protoplast-mediated transformation has been described for Japonica-types and Indica-types (Zhang et al. Plant Cell Rep 7: 379-384 (1988); Shimamoto et al. Nature 338: 274-277 (1989); Datta et al. Biotechnology 8: 736-740 (1990)). Both types are also routinely transformable using particle bombardment (Christou et al. Biotechnology 9: 957-962 (1991)). Furthermore, WO 93/21335 describes techniques for the transformation of rice via electroporation.

Patent Application EP 0 332 581 describes techniques for the generation, transformation and regeneration of Pooideae protoplasts. These techniques allow the transformation of Dactylis and wheat. Furthermore, wheat transformation has been described by Vasil et al. (Biotechnology 10: 667-674 (1992)) using particle bombardment into cells of type C long-term regenerable callus, and also by Vasil et al. (Biotechnology 11:1553-1558 (1993)) and Weeks et al. (Plant Physiol. 102: 1077-1084 (1993)) using particle bombardment of immature embryos and immature embryo-derived callus. One representative technique for wheat transformation involves the transformation of wheat by particle bombardment of immature embryos and includes either a high sucrose or a high maltose step prior to nucleic acid delivery. Prior to bombardment, any number of embryos (0.75-1 mm in length) are plated onto MS medium with 3% sucrose (Murashiga & Skoog, Physiologia Plantarum 15: 473-497 (1962)) and 3 mg/l 2,4-D for induction of somatic embryos, which is allowed to proceed in the dark. On the chosen day of bombardment, embryos are removed from the induction medium and placed onto the osmoticum (i.e. induction medium with sucrose or maltose added at the desired concentration, typically 15%). The embryos are allowed to plasmolyze for 2-3 hours and are then bombarded. Twenty embryos per target plate is typical, although not critical. An appropriate gene-carrying plasmid (such as pCIB3064 or pSOG35) is precipitated onto micrometer size gold particles using standard procedures. Each plate of embryos is shot with the DuPont BIOLISTICS® helium device using a burst pressure of about 1000 psi using a standard 80 mesh screen. After bombardment, the embryos are placed back into the dark to recover for about 24 hours (still on osmoticum). After 24 hrs, the embryos are removed from the osmoticum and placed back onto induction medium where they stay for about a month before regeneration. Approximately one month later the embryo explants with developing embryogenic callus are transferred to regeneration medium (MS+1 mg/liter NAA, 5 mg/liter GA), further containing the appropriate selection agent (10 mg/l basta in the case of pCIB3064 and 2 mg/l methotrexate in the case of pSOG35). After approximately one month, developed shoots are transferred to larger sterile containers known as “GA7s” which contain half-strength MS, 2% sucrose, and the same concentration of selection agent.

Transformation of monocotyledons using Agrobacterium has also been described. See, WO 94/00977 and U.S. Pat. No. 5,591,616, both of which are incorporated herein by reference. See also, Negrotto et al., Plant Cell Reports 19: 798-803 (2000), incorporated herein by reference.

For example, rice (Oryza sativa) can be transformed with Agrobacterium for generating transgenic plants. Various rice cultivars can be used (Hiei et al., 1994, Plant Journal 6:271-282; Dong et al., 1996, Molecular Breeding 2:267-276; Hiei et al., 1997, Plant Molecular Biology, 35:205-218). Those skilled in the art will appreciate that the various media constituents described below may be either varied in quantity or substituted. In an exemplary protocol, embryogenic responses are initiated and/or cultures are established from mature embryos by culturing on MS-CIM medium (MS basal salts, 4.3 g/liter; B5 vitamins (200×), 5 ml/liter; Sucrose, 30 g/liter; proline, 500 mg/liter; glutamine, 500 mg/liter; casein hydrolysate, 300 mg/liter; 2,4-D (1 mg/ml), 2 ml/liter; adjust pH to 5.8 with 1 N KOH; Phytagel, 3 g/liter). Either mature embryos at the initial stages of culture response or established culture lines are inoculated and co-cultivated with the Agrobacterium tumefaciens strain LBA4404 (Agrobacterium) containing the desired vector construction. Agrobacterium is cultured from glycerol stocks on solid YPC medium (100 mg/L spectinomycin and any other appropriate antibiotic) for about 2 days at 28° C. Agrobacterium is re-suspended in liquid MS-CIM medium. The Agrobacterium culture is diluted to an OD600 of 0.2-0.3 and acetosyringone is added to a final concentration of 200 uM. Acetosyringone is added before mixing the solution with the rice cultures to induce Agrobacterium for DNA transfer to the plant cells. For inoculation, the plant cultures are immersed in the bacterial suspension. The liquid bacterial suspension is removed and the inoculated cultures are placed on co-cultivation medium and incubated at 22° C. for two days. The cultures are then transferred to MS-CIM medium with Ticarcillin (400 mg/liter) to inhibit the growth of Agrobacterium. For constructs utilizing the PMI selectable marker gene (Reed et al., In Vitro Cell. Dev. Biol.-Plant 37:127-132), cultures are transferred to selection medium containing Mannose as a carbohydrate source (MS with 2% Mannose, 300 mg/liter Ticarcillin) after 7 days, and cultured for 3-4 weeks in the dark. Resistant colonies are then transferred to regeneration induction medium (MS with no 2,4-D, 0.5 mg/liter IAA, 1 mg/liter zeatin, 200 mg/liter timentin 2% Mannose and 3% Sorbitol) and grown in the dark for 14 days. Proliferating colonies are then transferred to another round of regeneration induction media and moved to the light growth room. Regenerated shoots are transferred to GA7 containers with GA7-1 medium (MS with no hormones and 2% Sorbitol) for 2 weeks and then moved to the greenhouse when they are large enough and have adequate roots. Plants are transplanted to soil in the greenhouse (To generation) grown to maturity, and the T1 seed is harvested.

The plants obtained via transformation with a nucleic acid sequence of the present invention can be any of a wide variety of plant species, including those of monocots and dicots. The expression of a nucleic acid encoding a cellulase enzyme in combination with other characteristics important for production and quality can be incorporated into plant lines through breeding. Breeding approaches and techniques are known in the art. See, for example, Welsh J. R., Fundamentals of Plant Genetics and Breeding, John Wiley & Sons, NY (1981); Crop Breeding, Wood D. R. (Ed.) American Society of Agronomy Madison, Wis. (1983); Mayo O., The Theory of Plant Breeding, Second Edition, Clarendon Press, Oxford (1987); Singh, D. P., Breeding for Resistance to Diseases and Insect Pests, Springer-Verlag, NY (1986); and Wricke and Weber, Quantitative Genetics and Selection Plant Breeding, Walter de Gruyter and Co., Berlin (1986).

In a representative protocol for the transformation of plastids, seeds of Nicotiana tabacum c.v. “Xanthienc” can be germinated seven per plate in a 1″ circular array on T agar medium and bombarded 12-14 days after sowing with 1 um tungsten particles (M10, Biorad, Hercules, Calif.) coated with DNA from plasmids pPH143 and pPH145 essentially as described (Svab, Z. and Maliga, P. (1993). PNAS 90, 913-917). Bombarded seedlings are incubated on T medium for two days after which leaves are excised and placed abaxial side up in bright light (350-500 nmol photons/m2/s) on plates of RMOP medium (Svab, Z., Hajdukiewicz, P. and Maliga, P. (1990) PNAS 87, 8526-8530) containing 500 ug/ml spectinomycin dihydrochloride (Sigma, St. Louis, Mo.). Resistant shoots appearing underneath the bleached leaves three to eight weeks after bombardment are subcloned onto the same selective medium, allowed to form callus, and secondary shoots isolated and subcloned. Complete segregation of transformed plastid genome copies (homoplasmicity) in independent subclones is assessed by standard techniques of Southern blotting (Sambrook et al., (1989) Molecular Cloning: A Laboratory Manual, Cold Spring Harbor Laboratory, Cold Spring Harbor). BamHI/EcoRI-digested total cellular DNA (Mettler, I. J. (1987) Plant Mol Biol Reporter 5, 346349) is separated on 1% Tris-borate (TBE) agarose gels, transferred to nylon membranes (Amersham) and probed with .sup.32P-labeled random primed DNA sequences corresponding to a 0.7 kb BamHI/HindIII DNA fragment from pC8 containing a portion of the rps 7/12plastid targeting sequence. Homoplasmic shoots are rooted aseptically on spectinomycin-containing MS/IBA medium (McBride, K. E. et al. (1994) PNAS 91, 7301-7305) and transferred to the greenhouse.

The genetic properties engineered into the transgenic seeds and plants described above can be passed on by sexual reproduction or vegetative growth and can thus be maintained and propagated in progeny plants. Generally, maintenance and propagation make use of known agricultural methods developed to fit specific purposes such as tilling, sowing or harvesting.

Use of the advantageous genetic properties of the transgenic plants and seeds can further be made in plant breeding. Depending on the desired properties, different breeding measures are taken. Suitable techniques are well known in the art, and include, but are not limited to, hybridization, inbreeding, backcross breeding, multi-line breeding, variety blend, interspecific hybridization, aneuploid techniques, etc. Thus, the transgenic seeds and plants can be used for the breeding of improved plant lines that, for example, increase the effectiveness of conventional methods of biomass processing.

Some aspects of the present invention are exemplified in greater detail below.

### EXAMPLES

### Example 1

### Potentiation of Activity of Enzyme Cocktail with Added Cellulolytic Enzymes

A microbial enzyme cocktail (Accellerase® 1000—Genencor™) (1, 5, or 15 mg/g), with and without 3 mg/g of a mixture of three enzymes—cellobiohydrolase I (CBHI), cellobiohydrolase II (CBHII), and endoglucanase (EG) (3 mg each enzyme/g cellulose)—was incubated with cellulose, and samples were taken at 0, 6, and 48 hrs.

At 6 hours, 5 mg/g cocktail with three enzymes was more efficient than three times the recommended dose of the cocktail alone (15 mg/g). At 48 hrs, the addition of the three enzymes (labeled as 3E) at least doubled the conversion of the cocktail alone at the given suboptimal cocktail concentrations. Further, a three-fold reduction of the cocktail with three enzymes was over 80% as active as the full dosage cocktail alone. Data in Table 1 is presented as percentages of normal where the value for 15 mg cocktail per g of cellulose at 48 hours incubation is considered normal and is set at 100%.

TABLE 1

| | 1 mg/g | 5 mg/g | 15 mg/g | 3E(3 mg/g) + | 3E(3 mg/g) + |
|---|---|---|---|---|---|
| hr | Acc | Acc | Acc | 1 mg/g Acc | 5 mg/g Acc |
| 0 | 0 | 0 | 0.8 | 1.9 | 1.9 |
| 6 | 4.37 | 14.7 | 44.7 | 30.0 | 57.19 |
| 48 | 12.4 | 36.47 | 100 | 63.2 | 83.09 |

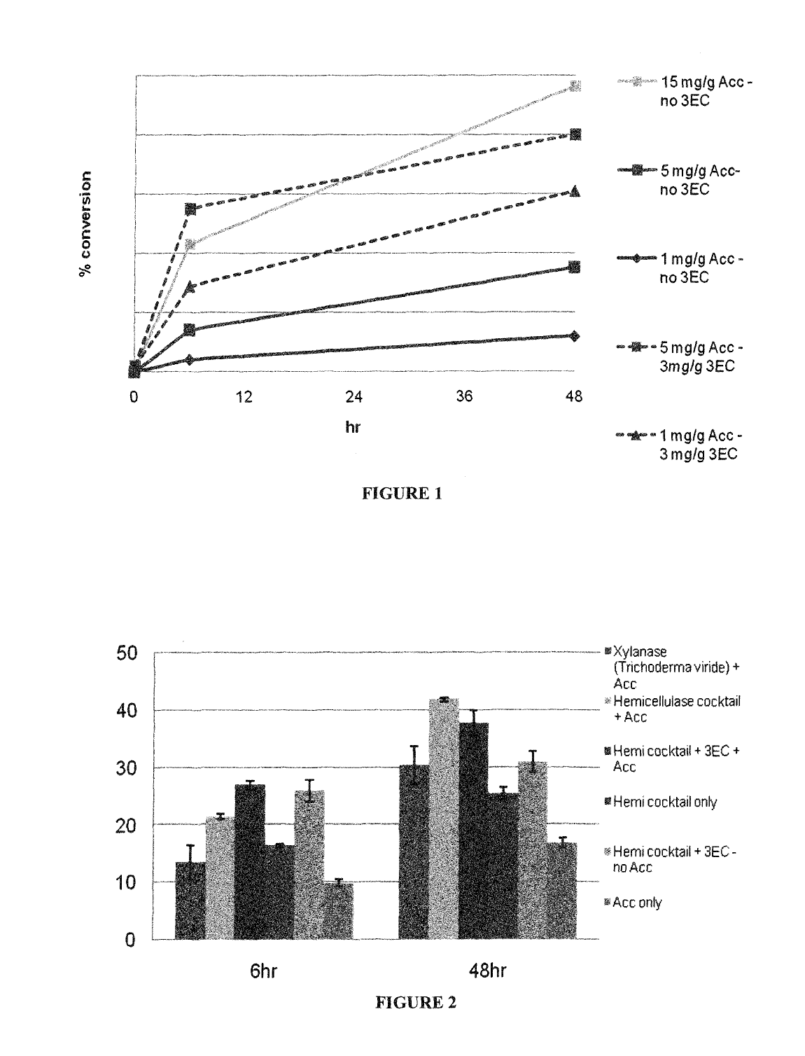

See also FIG. 1, which presents data of the conversion of acid-steam exploded sugarcane bagasse to glucose by an enzyme cocktail (Accellerase® 1000) plus a set of three enzymes: CBH I+CBH II+EG (labeled as 3EC for three-enzyme mixture). Addition of 3 mg/g of the set of three enzymes improved the percent conversion obtained at 6 and 48 hours with the use of 1 and 5 mg/g enzyme cocktail.

FIG. 2 reports the release of glucose at 6 hours and 48 hours from non-pretreated corn seed fiber catalyzed by different mixtures of Accellerase® 1000, a hemicellulase cocktail, xylanase and 3EC mixtures of hemicellulolytic enzymes.

### Example 2

### Saccharification of Cob/Stover and Switchgrass

Saccharification analysis of mild aqueous ammonia pre-treated cob/stover (a combination of field stover without cob material+corn cobs collected from different fields) and switchgrass was performed using a commercial cellulose cocktail (Accellerase® 1000—Genencor™) at various concentrations, in combination with cellulase enzymes. The cellulase enzyme classes included cellobiohydrolase I (CBHI), endoglucanase (EG), xylanase and beta-glucosidase (BG). These enzymes can also be expressed in plant systems.

High cellulose conversion was seen with a cocktail dosage greater than 25 mg per g cellulose. A significant increase in cellulose conversion occurred when enzymes are supplemented to the cellulose conversion reaction, where cocktail concentration is smaller than 25 mg per g cellulose.

Regarding the supplementary effect of a single enzyme, the cellulase classes (CBHI or EG) or xylanase resulted in enhanced saccharification with the cob/stover substrate when they are added to the cocktail, while under the same conditions there was no apparent enhancement in cellulose conversion with switchgrass.

Saccharification of 0.25 mm-ground 7% aqueous ammonia pretreated cob/stover and switchgrass was evaluated with combinations of Cellobiohydrolase I (CBH I), Endoglucanase (EG), Xylanase (Xyl), Beta-glucosidase(bG), and cocktail, at different levels of each enzyme. Conditions used throughout this Example: 1% milled solids (˜0.40% cellulose), incubation at 40° C. for 48 hours, at pH 4.8.

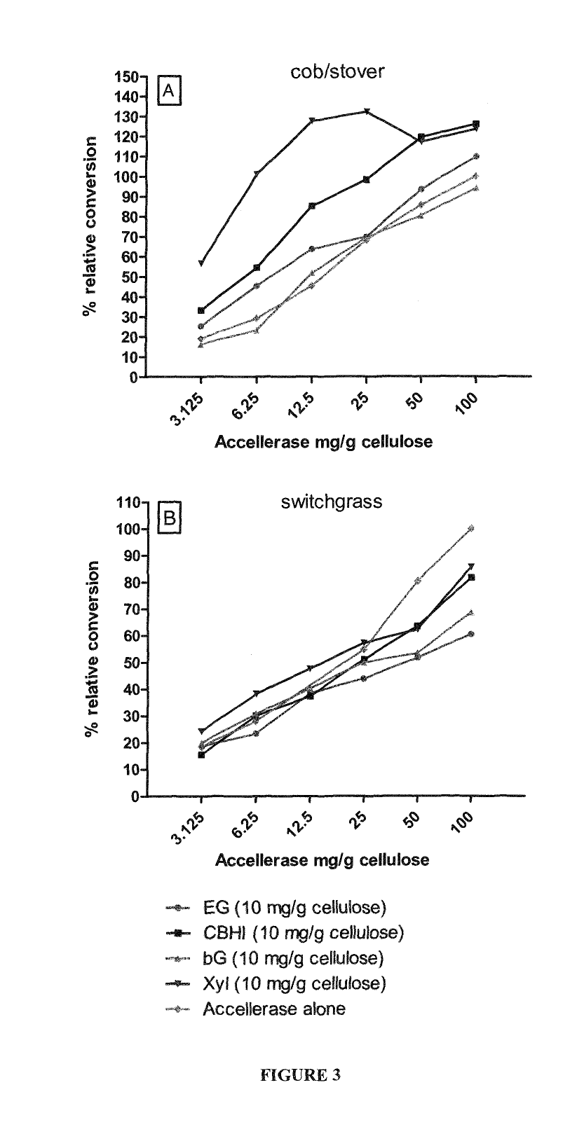

Cellulose conversion with respect to each component, for both cob/stover and switchgrass substrates, is shown in FIG. 3. The results suggest that the cocktail is more effective on steam-exploded pre-treated substrates than on mild ammonia treated substrates. Under the conditions tested here with the cob/stover substrate, the impact of supplementary cellulases increases as the concentration of the cocktail decreases, and switchgrass samples followed a similar trend.

At 25 mg cocktail, various combinations of cellulases increased the conversion from 68.3% (cocktail only) to a maximum of 132.1% of the conversion level achieved with 100 mg cocktail alone, indicating that the cellulase enzymes enhanced cellulose conversion by 63.8%. At 12.5 mg cocktail, the range of conversion was 45.3% (cocktail only) up to 130.3% of the level achieved with 100 mg cocktail alone, indicating that cellulase enzymes enhanced cellulose conversion by 85%. At 6.3 mg cocktail, the range was 29.2%-119.7% of the level achieved with 100 mg cocktail alone, indicating that cellulase enzymes enhanced cellulose conversion by 90.5%. In the absence of cocktail, 0-21.2% conversion was observed, indicating that various combinations of the cellulase enzymes alone reached the upper limit of 21.2% conversion.

The supplementary enzymes (Xyl or CBHI) substantially enhanced the conversion of cob/stover in combination with the cocktail compared to cocktail alone while EG or bG resulted in no appreciable increase in cellulose conversion (FIG. 3A). Single enzyme additions resulted in no appreciable increase in cellulose conversion with switch grass (FIG. 3B).

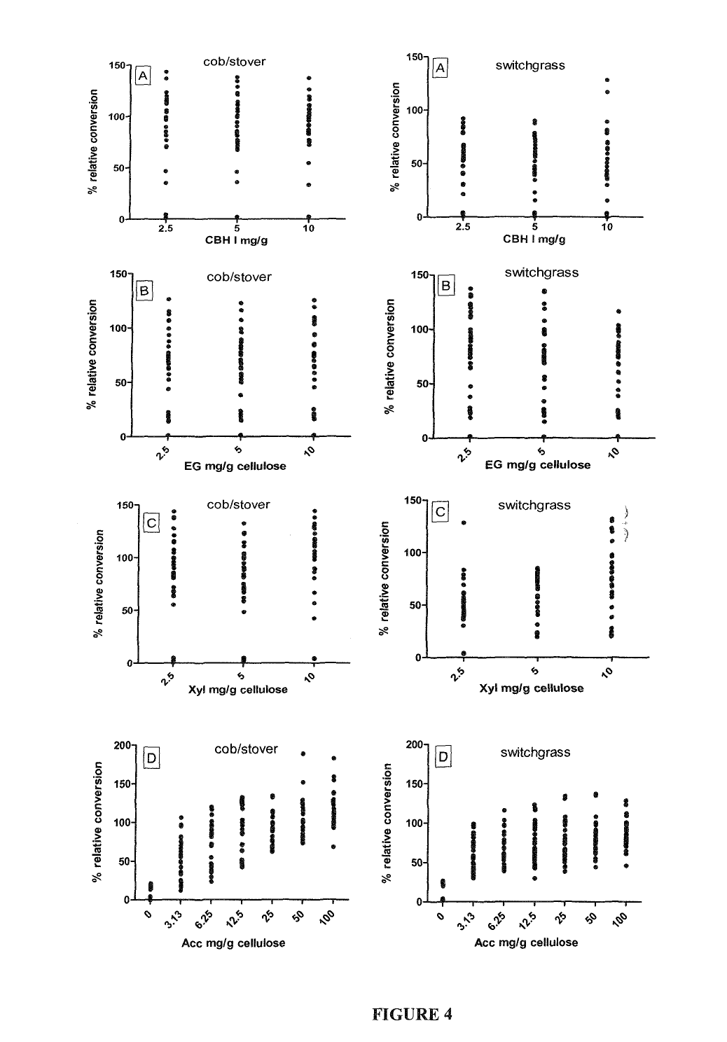

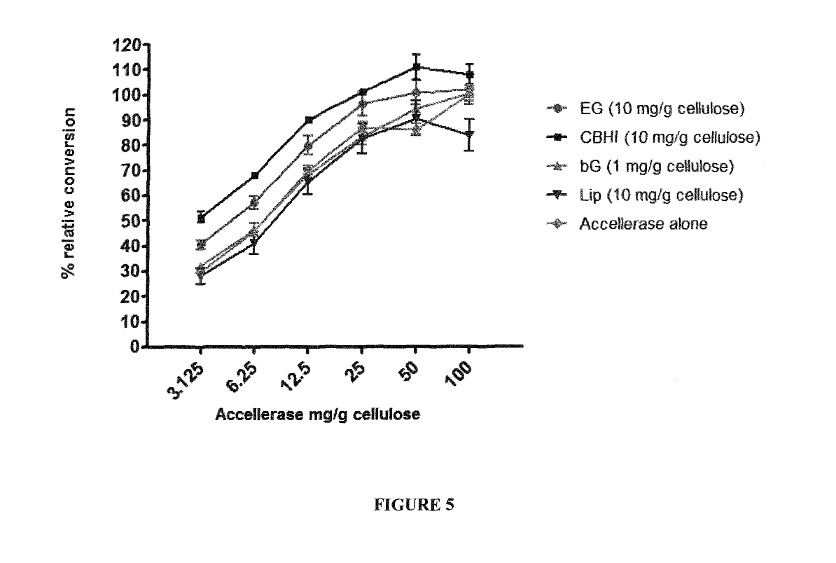

As a comparison, FIG. 4A-D presents the cellulose conversion, normalized to level achieved with 100 mg/g cocktail (Accellerase® 1000), with respect to each component of the factorial screen: CBH I(A), EG(B), Xyl(C) or cocktail (D) with different combinations of enzymes for both cob/stover and switchgrass.

Table 2 illustrates the impact of combinations of two different supplementary enzymes on cellulose conversion at a single concentration of cocktail (25 mg/g). Two cellulose enzymes were added (1:1 mass ratio) in the presence of 25 mg/g cocktail, and compared to the effect of a single enzyme addition at the same cocktail concentration for both cob/stover and switchgrass substrates. The combinations of two supplementary enzymes (e.g. Xyl and EG) with cocktail significantly enhanced cellulose conversion over the saccharification levels achieved with cocktail supplemented with a single cellulase enzyme for both cob/stover and switchgrass.

Substrate inhibition due to excess addition of cellulase enzyme was not observed with the pre-treated cob/stover and switchgrass. This is contrary to what was observed in steam-exploded bagasse hydrolysis.

Different doses of two supplementary cellulase enzymes (1:1 mass ratio) were tested in the presence of 25 mg/g cocktail to determine the conditions for the most efficient saccharification of aqueous ammonia treated cob/stover and switchgrass. Table 2 illustrates the cellulose conversion with the two supplementary cellulases at high, medium or low level. Overall, the supplement of high dose of cellulase enzymes (10 mg/g) resulted in an enhanced saccharification while the low dose (2.5 mg/g) resulted in no appreciable enhancement.

TABLE 2
Cumulative effect of two cellulases (in 1:1 ratio) on the ability of
Accellerase ® 1000 to degrade aqueous ammonia treated cob/
stover and switchgrass, compared to the effect observed
with the addition of just one cellulose enzyme.

| | Dosage | | |
|---|---|---|---|
| enzymes | low | medium | high |

Cob/Stover

| | | | |
|---|---|---|---|
| EG, Xyl, Acc | 105.6 | 114.2 | 147.9 |
| Xyl, Acc | 92.7 | 97.7 | 132.1 |
| CBHI, Xyl, Acc | 81.6 | 99.2 | 110.4 |
| CBHI, Acc | 76.9 | 81.7 | 98.3 |
| CBHI, EG, Acc | 80.1 | 87.4 | 90.5 |
| Acc (25 mg/g) | 80.0 | NA | NA |
| EG, Acc | 69.0 | 63.7 | 69.8 |
| bG, Acc | 61.9 | 67.0 | 69.4 |

Switchgrass

| | | | |
|---|---|---|---|
| EG, Xyl, Acc | 73.7 | 97.1 | 116.1 |
| CBHI, Xyl, Acc | 53.6 | 59.4 | 70.5 |
| Xyl, Acc | 46.3 | 56.9 | 57.2 |
| Acc | 54.8 | NA | NA |
| CBHI, Acc | 47.2 | 47.0 | 51.1 |
| bG, Acc | 39.0 | 54.0 | 50.0 |
| EG, Acc | 47.3 | 45.9 | 43.9 |
| CBHI, EG, Acc | 44.3 | 38.9 | 43.3 |

Note:
Cellulose conversion is normalized to level achieved with 100 mg/g Accellerase ®.

### Conclusions.

Cob/stover: based on the data presented here, to achieve the same level of conversion as observed with the maximum dose of cocktail at 100 mg/g cellulose, the supplement of each 10 mg of Xylanase alone or the combinations of Xylanase and EG or Xylanase and CBH1 to ˜25 mg cocktail/g cellulose appeared equivalent. In other words, the supplement of Xylanase alone or together with EG or CBH1 would lower the requisite cocktail dose from 100 mg/g cellulose to ˜25 mg/g cellulose.

Switchgrass: based on the data presented here, to achieve the same level of conversion as observed with the maximum dose of cocktail at 100 mg/g cellulose, a supplement of 10 mg Xyl+EG per g cellulose would lower the requisite cocktail dose from 100 mg/g cellulose to ˜25 mg/g.

### Materials and Methods

### Substrate and Enzymes

Cob/stover substrate: 75% corncob/25% stover pretreated with 7% aqueous ammonia. 60-mesh (˜0.25 mm average particle size) ground. Estimated: glucan 40%, 26.5% xylan.
Switchgrass substrate: 7% aqueous ammonia treated, 60-mesh (˜0.25 mm average particle size) ground. Estimated: glucan ˜40%, ˜26.5% xylan.
Accellerase® 1000 (Sample Batch #1600794133, Genencor, Rochester, N.Y. 14618, USA)
   Note: Accellerase® was clarified by centrifugation prior to use.
beta Glucosidase (bG, sample batch #50502, E.C #3.2.1.21, Megazyme, Wicklow, Ireland), beta Xylanase M1 (Xyl, sample batch #20502, Megazyme, Wicklow, Ireland)
Fungal cellobiohydrolase I (CBHI, SEQ ID NO:1) and Endoglucanase (EG, SEQ ID NO:4) were expressed in Aspergillus niger, and were purified at SBI. Purification involved preliminary clarification and concentration by ethanol precipitation, followed by further purification using Phenyl Sepharose followed by Anion exchange for CBH I, or Octyl Sepharose for EG. Enzymes were desalted into 5 mM Ammonium acetate, pH 7.4, and lyophilized. Enzymes were resuspended to ˜5 mg/ml in 50 mM Na-Acetate, pH 5, 100 mM NaCl, 0.02% azide, and stored at 4° C.
All possible combinations of CBHI, EG, Xyl, bG and Accellerase® were tested at 5 different levels of each cellulase.
For CBHI, bG, Xyl, and EG, the doses were 0, 2.5, 5, 7.5 and 10 mg/g cellulose. For Accellerase®, 0, 3.13, 6.3, 12.5, 25, 50 and 100 mg/g cellulose.

### Saccharification Reactions

Enzymes were dispensed using a Biomek robotic system.
Standard saccharification reaction conditions were as follows:
   1% solids (0.65% cellulose) in 50 mM Sodium Acetate, 0.02% Na-azide, pH 4.8, in 96 well, flat-bottom (Costar) microtiter plates.
   Temperature at 40° C.
   Agitation at 200 rpm with a 3.5 mm diameter steel BB in each well.
   Unless otherwise indicated, cellulose conversion was measured after 48 hr incubation (single time point only)
   Note: Accellerase was clarified by centrifugation prior to use.
Reactions were stopped by the addition of 20 ul of 1M Sodium Carbonate pH 10 for every 150 ul of reaction. Glucose concentrations were assayed using a Glucose Oxidase kit (Pointe Scientific, Fisher cat#G7521) and results are presented as a percent of the theoretical maximum glucose yield based upon the cellulose concentration in the reaction.

### Data Analysis

Data was formulated as the relative yield of saccharification to the yield achieved with 100 mg of Accellerase® alone.
Prior to analysis, significant outliers were dropped based upon visual inspection of the reaction plates (e.g., those wells with visible evaporation problems).
The data presented here are the average of triplicates in each treatment.

### Example 3

### Saccharification of Bagasse

Saccharification analysis of steam-exploded sugarcane bagasse was performed using a commercial cellulase cocktail at various concentrations (Accellerase® 1000—Genencor) in combination with one or two microbially expressed enzymes that can be expressed in plant systems. These enzyme classes include cellobiohydrolase I (CBHI), endoglucanase (EG), Lignon Peroixidase (LiP) and beta-glucosidase (BG). All possible combinations of CBHI, EG, LiP, bG and cocktail were screened at 5 different levels of each cellulase.

Saccharification of 0.25 mm-ground steam-exploded pretreated bagasse was evaluated with combinations of Cellobiohydrolase I (CBH I), Endoglucanase (EG), Lignin Peroxidase (LiP), Beta-glucosidase(bG), and cocktail, at different levels of each enzyme. For CBHI, LiP, and EG, the doses were 0, 2.5, 5, 7 and 10 mg/g cellulose. For bG the doses were 0, 0.06, 0.25 and 1 mg/g cellulose. For the cocktail, 0, 3.13, 6.3, 12.5, 25, 50 and 100 mg/g cellulose was used. Data were generated using the conditions: 1% milled solids (˜0.65% cellulose), incubation at 40° C. for 48 hours, at pH 4.8.

High cellulose conversion was seen with a cocktail dosage greater than 25 mg per g cellulose. A significant increase in cellulose conversion occurs when enzymes are supplemented to the cellulose conversion reaction, where cocktail concentration is smaller than 25 mg per g cellulose.

Regarding the supplementary effect of a single enzyme, the cellulase classes (CBHI or EG) resulted in enhanced saccharification when they are added to the cocktail, while BG and LiP resulted in no apparent enhancement in cellulose conversion.

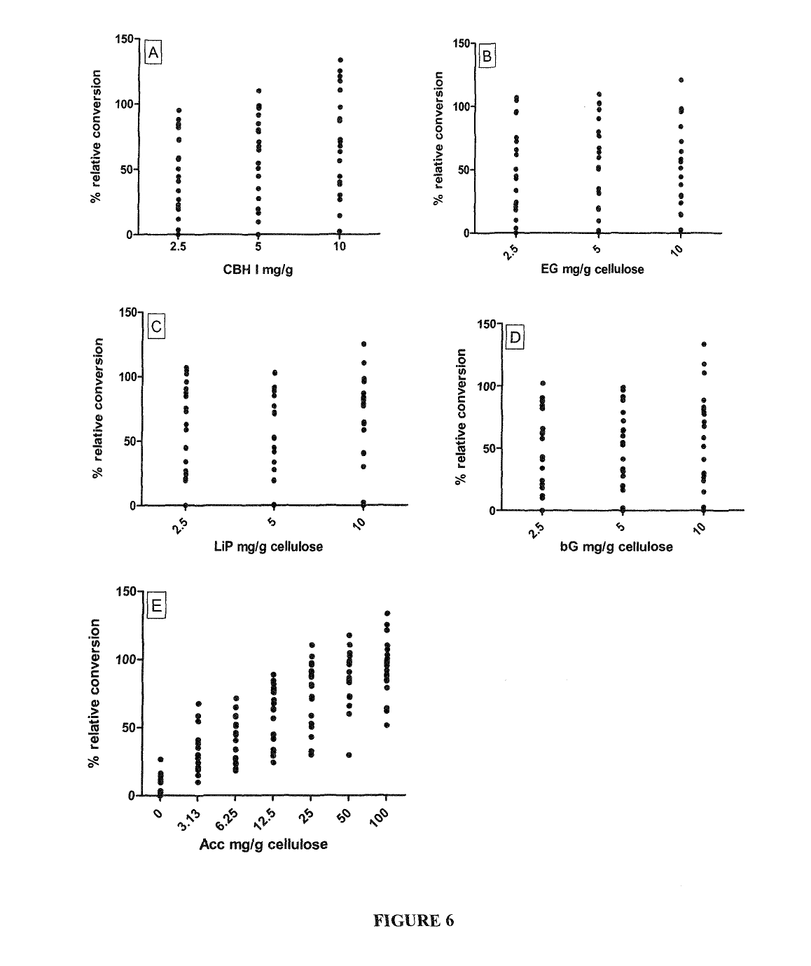

Cellulose conversion with respect to each component is shown in FIG. 5. The various enzyme combinations resulted in a wide spectrum of saccharification yield, in which the cocktail was the major driving force for increased cellulose conversion. See FIG. 6.

Under the conditions tested here, the impact of supplementary cellulases increases as the concentration of cocktail decreases. At 12.5 mg cocktail, various combinations of cellulases increased conversion from 70% (Accellerase® only) to a maximum of 88.5% of the conversion level achieved with 100 mg Accellerase® alone, indicating that the cellulase enzymes enhanced cellulose conversion by 18.5%. At 6.3 mg cocktail, the range of conversion was 46% (Accellerase® only) up to 71% of level achieved with 100 mg cocktail alone, indicating that JBP enhanced cellulose conversion by 25.1%. At 113 mg cocktail, the range was 29.7%-67.6% of the level achieved with 100 mg cocktail alone, indicating that JBP enhanced cellulose conversion by 37.9%. In the absence of cocktail, 0-26.6% conversion was observed, indicating that various combinations of cellulases alone reached the upper limit of 26.6% conversion under these conditions.

The supplementary enzymes (CBHI or EG) substantially enhanced the conversion in combination with cocktail compared to the cocktail alone. BG and LiP resulted in no appreciable increase in cellulose conversion.

Table 3 illustrates the impact of combinations of two different supplementary enzymes on cellulose conversion at a single concentration of cocktail (25 mg/g). Two cellulose enzymes were added (1:1 mass ratio) in the presence of 25 mg/g cocktail, and compared to the effect of a single enzyme addition at the same cocktail concentration.

The combinations of two supplementary enzymes resulted in a small appreciable difference compared to the single supplementary enzyme in terms of cellulose conversion. In some instances, supplement of two enzymes to cocktail resulted in a decrease in saccharification yield (i.e., EG+bG+Acc), indicating that too much total enzyme can inhibit overall saccharification.

Different doses of two supplementary cellulase enzymes (1:1 mass ratio) were tested in the presence of 25 mg/g cocktail to determine the conditions for the most efficient saccharification of steam-exploded bagasse. Table 3 illustrates the cellulose conversion with the two supplementary cellulases at high, medium and low level. Overall, the supplement of high dose of cellulase enzymes (10 mg/g) resulted in an enhanced saccharification while the low dose (2.5 mg/g) resulted in no appreciable enhancement.

TABLE 3
Cumulative effect of two cellulases (in 1:1 ratio) on the ability of
Accellerase ® 1000 to degrade STX bagasse, compared to the effect
observed with the addition of just one cellulase enzyme.
Bagasse

| | Dosage | | |
|---|---|---|---|
| enzymes | low | medium | high |
| CBHI, bG, Acc | 88.0 | 91.4 | 110.3 |
| CBHI, Acc | 89.9 | 90.5 | 101.0 |
| EG, LiP, Acc | 90.6 | 91.5 | 97.1 |
| EG, Acc | 86.6 | 91.6 | 96.3 |
| bG, Acc | 84.7 | 87.2 | 86.3 |
| LiP, Acc | 85.8 | 84.1 | 82.5 |
| Acc | 82.0 | NA | NA |
| CBHI, LiP, Acc | 63.5 | 72.5 | 87.0 |
| CBHI, EG, Acc | 50.2 | 75.2 | 79.9 |
| bG, LiP, Acc | 50.9 | 55.2 | 81.4 |
| EG, bG, Acc | 43.0 | 32.6 | 29.9 |

NOTE:
Cellulose conversion is normalized to level achieved with 100 mg/g Accellerase ®.

Based on the data presented here, to achieve the same level of conversion as observed with the maximum dose of cocktail at 100 mg/g cellulose, the supplement of each 10 mg of CBHI and EG/g cellulose to ˜25 mg cocktail/g cellulose appeared pertinent. In other words, the supplement of CBHI and EG (each 10 mg/g cellulose) would lower the requisite cocktail dose from 100 mg/g cellulose to ˜25 mg/g cellulose.

### Materials and Methods

### Substrate and Enzymes

Substrate: Pretreated steam exploded bagasse ground to 60-mesh (=˜0.25 mm average particle size) (Glucan 65%, 4.7 xylan).
Accellerase® 1000 (Sample Batch #1600794133, Genencor, Rochester, N.Y. 14618, USA)
   Note: Accellerase® was clarified by centrifugation prior to use.
Lignin Peroxidase (LiP, sample batch #42603, CAS #42613-30-9, Sigma, St. Louis, Mo. 63103, USA) and beta Glucosidase (bG, sample batch #50502, E.C #3.2.1.21, Megazyme, Wicklow, Ireland)
Fungal cellobiohydrolase I (CBHI, SEQ ID NO:1) and Endoglucanase (EG, SEQ ID NO:4) were expressed in Aspergillus niger, and were purified at SBI. Purification involved preliminary clarification and concentration by ethanol precipitation, followed by further purification using Phenyl Sepharose followed by Anion exchange for CBHI, or Octyl Sepharose for Ea Enzymes were desalted into 5 mM Ammonium acetate, pH 7.4, and lyophilized. Enzymes were resuspended to ˜5 mg/ml in 50 mM Na-Acetate, pH 5, 100 mM NaCl, 0.02% azide, and stored at 4 C.

### Saccharification Reactions

Enzymes were dispensed using a Biomek robotic system.
Standard saccharification reaction conditions were as follows:
   1% solids (0.65% cellulose) in 50 mM Sodium Acetate, 0.02% Na-azide, pH 4.8, in 96 well, flat-bottom (Costar) microtiter plates.
   Temperature at 40° C.
   Agitation at 200 rpm with a 3.5 mm diameter steel BB in each well.
   Unless otherwise indicated, cellulose conversion was measured after 48 hr incubation (single time point only)
   Note: Accellerase® was clarified by centrifugation prior to use.
Reactions were stopped by the addition of 20 μl of 1M Sodium Carbonate pH 10 for every 150 μl of reaction. Glucose concentrations were assayed using a Glucose Oxidase kit (Pointe Scientific, Fisher cat#G7521) and results are presented as a percent of the theoretical maximum glucose yield based upon the cellulose concentration in the reaction.

### Data Analysis

Data is formulated as the relative yield of saccharification to the yield achieved with 100 mg of cocktail alone.
Prior to analysis, significant outliers were dropped based upon visual inspection of the reaction plates (e.g., those wells with visible evaporation problems).
The data presented here are the average of triplicates in each treatment.

### Example 4

### Addition of Cellulase Enzymes Expressed in Plants

One or more cellulase enzymes such as CBH I, CBH II and/or EG are expressed from a heterologous nucleic acid stably introduced into a plant. The enzyme-expressing plants are used as the biomass or added to biomass intended to be converted to fermentable sugars with the use of an enzyme cocktail. The presence of one or more of these enzymes potentiates the activity (efficiency and/or yield) of a microbial cellulase cocktail. This allows a lower dose of the microbial cellulase cocktail to be used, thereby lowering process costs.

CBH I may be, for example, the amino acid sequence of SEQ ID NO:1, or a protein having at least 80, 90, 95 or 99% identity thereto. CBH II may be, for example, the amino acid sequence of SEQ ID NO:2, or a protein having at least 80, 90, 95 or 99% identity thereto. EG may be, for example, the amino acid sequence of SEQ ID NO:3, or a protein having at least 80, 90, 95 or 99% identity thereto, or the amino acid sequence of SEQ ID NO:4, or a protein having at least 80, 90, 95 or 99% identity thereto.

> CBH I
(SEQ ID NO: 1)
msalnsfnmyksalilgsllatagaqqigtytaethpslswstcksggscttnsgaitldanwrwvhgvntstncytgntwntaicdtdas
caqdcaldgadysgtygittsgnslrlnfvtgsnvgsrtylmadnthyqifdllnqeftftvdvshlpcglngalyfvtmdadggvskyp
nnkagaqygvgycdsqcprdlkfiagqanvegwtpssnnantglgnhgaccaeldiweansisealtphpcdtpglsvcttdacggty
ssdryagtcdpdgcdfnpyrlgvtdfygsgktvdttkpitvvtqfvtddgtstgtlseirryyvqngvvipqpsskisgvsgnvinsdfcd
aeistfgetasfskhgglakmgagmeagmvlvmslwddysvnmlwldstyptnatgtpgaargscpttsgdpktvesqsgssyvtfs
dirvgpfnstfsggsstggsstttasgttttkasststsststgtgvaahwgqcggqgwtgpttcasgttctvvnpyysqcl

A 19-amino acid signal sequence, indicated in underline, is at the N-terminal portion of the mature CBH I protein in this construct. See also Genbank Accession No. CBX74419, which is a CBH I protein having an alternative signal sequence at the N-terminus.

>CBH II
(SEQ ID NO: 2)
mvvgilatlatlatlaasvpleerqscssvwgqcggqnwagpfccasgstcvysndyysqclpgtassssstrassttsrvssatstrsssst
pppassttpappvgsgtatysgnpfagvtpwansfyasevstlaipsltgamataaaavakvpsfmwldtldktplmsstlsdiraank
aggnyagqfvvydlpdrdcaaaasngeysiadggvakyknyidtirgivttfsdvrillviepdslanlvtnlatpkcsnaqsayleciny
aitqlnlpnvamyldaghagwlgwpanqdpaaqlfanvyknasspravrglatnvanynawnittppsytqgnavyneklyihalg
pllanhgwsnaffitdqgrsgkqptgqlewgnwcnavgtgfgirpsantgdslldsfvwikpggecdgtsnssaprfdyhcasadalq
papqagswfqayfvqlltnanpsfl

A 19-amino acid signal sequence, indicated in underline, is at the N-terminal portion of the Mature CBH II protein in this construct. See also Genbank Accession No. CBX74420, which is a CBH II protein having an alternative signal sequence at the N-terminus.

> EG
(SEQ ID NO: 3)
(Genbank Accession No. CBX74421)
matqgaldsavtalqsaittfsgarqdgaktsgftsaqytalinsakadkegvrtsangddvspveywvnssvlgafnaaitalenasgqs
aidaaylaliqagktfndakrhgttpdrtalnnaitaavnakngvqtaadkdqaslgsswatgaqfnalntaidsatavknnanatkasvd
taaaslnaaiatfttavtnngpgtqtfrditaaqlvaeikigwnlgnsldahngfpanptvdqmergwgnpattkanitalknagfnairip
vswtkaasgapnytirtdwmtrvkeivnyavdndmyiilnthhdedvltfmnsnaaagkaafqklweqiaaafkdyneklifeglne
prtpgssnewnggtdeernnlnsyypifvntvrssggnngkrilminpyaasmeavamnaltlpadsaanklivsfhsyqpynfaln
kdssintwsssssgdtspitgpidryynkfvsqgipviigefgamnknneavraqwaeyyvsyaqskgikcfwwdngvtsgsgelfg
llnrtnntftynallngmmsgtggtvptpptppatptppttitgnlgtyqfgtqedgvspnytqavwelsgtnlttakttgaklvlvfttapn
asmhfvwqgpanslwwnekeilgntgnpsatgvtwnsgtktltipltansvkdysvftaqpslriiiayynggnvndlgivsanltq
> EG
(SEQ ID NO: 4)
mkslfalslfaglsvaqnaawaqcggngwtgsktcvsgykctvvnewysqcipgtaeeptttlktttgggstptgtpgngkflwvgtne
aggefgegslpgtwgkhfifpdpaavdtlisqgynafrvqlrmertnpssmtgpfdtaylknlttivdhitgkganvildphnygryfdk
iitstsdfqtwwknfatqfksnskvifdtnneyntmdqtlvlnlnqaaingiraagatqtifvegnqwsgawswpdvndnmkaltdpl
dkivyemhqyldsdssgtspncvsttigvervkaatewlrknkkigmigelaggpndtcktavknmldylkensdvwkgvtwwaa
gpwwadymfsfeppsgtgyqyynsllktyi

Although the foregoing invention has been described in some detail by way of illustration and example for purposes of clarity and understanding, it will be clear to those of skill in the art that certain changes and modifications may be practiced within the scope of the appended claims.

1. A method of potentiating enzymatic saccharification of a cellulosic biomass using a microbial enzyme cocktail, said method comprising combining the microbial enzyme cocktail with at least one cellulase enzyme.

2. The method of claim 1, wherein the microbial enzyme cocktail is provided at a sub-maximum dose.

3. The method of claim 1, wherein the cellulase enzyme is selected from the group consisting of cellobiohydrolase I, cellobiohydrolase II and endoglucanase.

4. The method of claim 1, wherein the microbial enzyme cocktail is an enzyme cocktail from Trichoderma reesei, Trichoderma longibrachiatum, or Saccharophagus degradans.

5. The method of claim 1, wherein the cellulase enzyme is provided by expression thereof by a transgenic plant comprising a heterologous nucleic acid encoding the same.

6. The method of claim 5, wherein the transgenic plant is selected from the group consisting of sorghum, maize, soybean, switchgrass and sugar cane.

7. The method of claim 5, wherein said biomass comprises said transgenic plant.

8. The method of claim 1, wherein said cellulosic biomass comprises sugarcane bagasse, corn seed fiber, corn stover, switchgrass, wood pulp, or straw of rice, wheat or barley.

9. The method of claim 1, wherein said cellulosic biomass is pretreated.

10. A method of producing fermentable sugars, comprising:
combining a cellulosic biomass with a composition comprising a microbial enzyme cocktail and at least one cellulase enzyme under conditions conducive to producing fermentable sugars therefrom,
to thereby produce fermentable sugars.

11. The method of claim 10, wherein the microbial enzyme cocktail is provided at a sub-maximum dose.

12. The method of claim 10, wherein the cellulosic biomass comprises plant material selected from the group consisting of sugarcane bagasse, corn seed fiber, corn stover, switchgrass, wood pulp, or straw of rice, wheat or barley.

13. The method of claim 10, wherein the cellulase enzyme is selected from the group consisting of cellobiohydrolase I, cellobiohydrolase II and endoglucanase.

14. The method of claim 10, wherein the microbial enzyme cocktail is an enzyme cocktail from Trichoderma reesei, Trichoderma longibrachiatum, or Saccharophagus degradans.

15. The method of claim 10, further comprising the step of fermenting the fermentable sugars to produce ethanol.

16. A method of producing fermentable sugars, said method comprising:
(a) providing a mixture of:
(i) a transgenic plant comprising a cellulase enzyme expressed from a heterologous nucleic acid encoding the same therein; and
(ii) a composition comprising a microbial enzyme cocktail, and
(b) providing conditions conducive to producing fermentable sugars from said mixture, to thereby produce said fermentable sugars.

17. The method of claim 16, wherein said transgenic plant expresses between 1 and 30 mg of said cellulase enzyme per gram of cellulose of said plant.

18. The method of claim 16, wherein said cellulase enzyme is selected from the group consisting of cellobiohydrolase I, cellobiohydrolase II and endoglucanase.

19. The method of claim 18, wherein said cellobiohydrolase I, cellobiohydrolase II or endoglucanase comprises a heterologous linker sequence.

20. The method of claim 16, wherein the transgenic plant is selected from the group consisting of sorghum, maize, soybean, switch grass and sugar cane.

21. The method of claim 16, wherein the microbial enzyme cocktail is an enzyme cocktail from Trichoderma reesei, Trichoderma longibrachiatum, or Saccharophagus degradans.

22. The method of claim 16, wherein the microbial enzyme cocktail is provided at a concentration of between 5 and 90 mg per gram of cellulose of said plant.

23. The method of claim 16, wherein the microbial enzyme cocktail is provided at a concentration of between 10 and 50 mg per gram of cellulose of said plant.


SEQUENCE LISTING (5 sequences)

SEQ ID NO: 1 - protein, 529 aa, Artificial
  Synthetic construct comprising CBH I
MSALNSFNMYKSALILGSLLATAGAQQIGTYTAETHPSLSWSTCKSGGSCTTNSGAITLDANWRWVHGVNTSTNCYTGNTWNTAICDTDASCAQDCALDGADYSGTYGITTSGNSLRLNFVTGSNVGSRTYLMADNTHYQIFDLLNQEFTFTVDVSHLPCGLNGALYFVTMDADGGVSKYPNNKAGAQYGVGYCDSQCPRDLKFIAGQANVEGWTPSSNNANTGLGNHGACCAELDIWEANSISEALTPHPCDTPGLSVCTTDACGGTYSSDRYAGTCDPDGCDFNPYRLGVTDFYGSGKTVDTTKPITVVTQFVTDDGTSTGTLSEIRRYYVQNGVVIPQPSSKISGVSGNVINSDFCDAEISTFGETASFSKHGGLAKMGAGMEAGMVLVMSLWDDYSVNMLWLDSTYPTNATGTPGAARGSCPTTSGDPKTVESQSGSSYVTFSDIRVGPFNSTFSGGSSTGGSSTTTASGTTTTKASSTSTSSTSTGTGVAAHWGQCGGQGWTGPTTCASGTTCTVVNPYYSQCL

SEQ ID NO: 2 - protein, 472 aa, Artificial
  Synthetic construct comprising CBH II
MVVGILATLATLATLAASVPLEERQSCSSVWGQCGGQNWAGPFCCASGSTCVYSNDYYSQCLPGTASSSSSTRASSTTSRVSSATSTRSSSSTPPPASSTTPAPPVGSGTATYSGNPFAGVTPWANSFYASEVSTLAIPSLTGAMATAAAAVAKVPSFMWLDTLDKTPLMSSTLSDIRAANKAGGNYAGQFVVYDLPDRDCAAAASNGEYSIADGGVAKYKNYIDTIRGIVTTFSDVRILLVIEPDSLANLVTNLATPKCSNAQSAYLECINYAITQLNLPNVAMYLDAGHAGWLGWPANQDPAAQLFANVYKNASSPRAVRGLATNVANYNAWNITTPPSYTQGNAVYNEKLYIHALGPLLANHGWSNAFFITDQGRSGKQPTGQLEWGNWCNAVGTGFGIRPSANTGDSLLDSFVWIKPGGECDGTSNSSAPRFDYHCASADALQPAPQAGSWFQAYFVQLLTNANPSFL

SEQ ID NO: 3 - protein, 715 aa, Artificial
  Synthetic construct comprising EG
MATQGALDSAVTALQSAITTFSGARQDGAKTSGFTSAQVTALINSAKADKEGVRTSANGDDVSPVEYWVNSSVLGAFNAAITALENASGQSAIDAAYLALIQAGKTFNDAKRHGTTPDRTALNNAITAAVNAKNGVQTAADKDQASLGSSWATGAQFNALNTAIDSATAVKNNANATKASVDTAAASLNAAIATFTTAVTNNGPGTQTFRDITAAQLVAEIKIGWNLGNSLDAHNGFPANPTVDQMERGWGNPATTKANITALKNAGFNAIRIPVSWTKAASGAPNYTIRTDWMTRVKEIVNYAVDNDMYIILNTHHDEDVLTFMNSNAAAGKAAFQKLWEQIAAAFKDYNEKLIFEGLNEPRTPGSSNEWNGGTDEERNNLNSYYPIFVNTVRSSGGNNGKRILMINPYAASMEAVAMNALTLPADSAANKLIVSFHSYQPYNFALNKDSSINTWSSSSSGDTSPITGPIDRYYNKFVSQGIPVIIGEFGAMNKNNEAVRAQWAEYYVSYAQSKGIKCFWWDNGVTSGSGELFGLLNRTNNTFTYNALLNGMMSGTGGTVPTPPTPPATPTPPTTITGNLGTYQFGTQEDGVSPNYTQAVWELSGTNLTTAKTTGAKLVLVFTTAPNASMHFVWQGPANSLWWNEKEILGNTGNPSATGVTWNSGTKTLTIPLTANSVKDYSVFTAQPSLRIIIAYYNGGNVNDLGIVSANLTQ

SEQ ID NO: 4 - protein, 382 aa, Artificial
  Synthetic construct comprising EG
MKSLFALSLFAGLSVAQNAAWAQCGGNGWTGSKTCVSGYKCTVVNEWYSQCIPGTAEEPTTTLKTTTGGGSTPTGTPGNGKFLWVGTNEAGGEFGEGSLPGTWGKHFIFPDPAAVDTLISQGYNAFRVQLRMERTNPSSMTGPFDTAYLKNLTTIVDHITGKGANVILDPHNYGRYFDKIITSTSDFQTWWKNFATQFKSNSKVIFDTNNEYNTMDQTLVLNLNQAAINGIRAAGATQTIFVEGNQWSGAWSWPDVNDNMKALTDPLDKIVYEMHQYLDSDSSGTSPNCVSTTIGVERVKAATEWLRKNKKIGMIGELAGGPNDTCKTAVKNMLDYLKENSDVWKGVTWWAAGPWWADYMFSFEPPSGTGYQYYNSLLKTYI

SEQ ID NO: 5 - protein, 6 aa, Artificial
  Endoplasmic reticulum targeting sequence
SEKDEL